(19) United States
(12) Patent Application Publication
(10) Pub. No.: US 2024/0368079 A1
(43) Pub. Date: Nov. 7, 2024

(54) PROCESS FOR THE PREPARATION OF OPTICALLY PURE ENANTIOMERS OF CYCLIC IMINIUM SALTS AND THEIR USE AS CATALYSTS

(71) Applicants: Centre National de la Recherche Scientifique, Paris (FR); Ecole Nationale Supérieure de Chimie de Rennes, Rennes (FR); Institut National des Sciences Appliquées, Rennes (FR); Université D'aix-Marseille, Marseille (FR); Universite de Rennes, Rennes (FR); University of California, San Diego, La Jolla, CA (US)

(72) Inventors: Marc MAUDUIT, Vitre (FR); Jennifer MORVAN, Pluméliau (FR); Jan LORKOWSKI, Poznan (PL); Nicolas VANTHUYNE, Marseille (FR); Rodolphe JAZZAR, Solana Beach, CA (US); Guy BERTRAND, Solana Beach, CA (US)

(21) Appl. No.: 18/688,703

(22) PCT Filed: Sep. 1, 2022
(86) PCT No.: PCT/EP2022/074330

§ 371 Date: Mar. 1, 2024

(30) Foreign Application Priority Data
Sep. 1, 2021 (EP) 21306194.8

(51) Int. Cl. C07D 207/20; B01J 31/22; C07D 471/08; C09K 11/06; H10K 85/30

(52) U.S. Cl. CPC C07D 207/20; B01J 31/2226; B01J 31/2273; B01J 31/2291; B01J 31/2295; C07D 471/08; C09K 11/06; H10K 85/344; H10K 85/371; B01J 2531/004; B01J 2531/16; B01J 2531/18; B01J 2531/822; C09K 2211/1007; C09K 2211/1029; C09K 2211/185; C09K 2211/188

(57) ABSTRACT

The present invention relates to a process for the preparation of an optically pure (+) or (−) enantiomer of an iminium salt having the formula (I), aid process comprising the following steps:
a) a reduction step of an iminium salt having the formula (II), said salt being in the form of a racemic mixture, in order to obtain a compound having the formula (III) in the form of a racemic mixture,
b) a step of chiral HPLC separation of the compound of formula (III) in the form of a racemic mixture, for obtaining an optically pure (+) or (−) enantiomer compound having the formula (IV), and
c) an oxidation step of the compound of formula (IV) for obtaining the compound of formula (I).

The present invention concerns a process for the preparation of optically pure enantiomers of cyclic iminium salts, as well as the corresponding optically pure enantiomers of cyclic iminium salts.

Since their discovery in early 1960s, N-heterocyclic carbenes (NHCs) have become inescapable ligands in transition-metal (TM) catalyzed transformations, in both academic and industrial research environments (N-Heterocyclic Carbenes: From Laboratory Curiosities to Efficient Synthetic Tools (Eds.: S. Díez-González), RSC Catalysis series, RSC Publishing: Cambridge, 2011). In part, this growing popularity has been attributed to their remarkable aptitude in generating more stable, yet very reactive catalysts. Not surprisingly, chiral variants of diaminocarbenes naturally emerged in early 1990s, and thanks to their unique and highly modular steric environment, they also rapidly became privileged stereo-directing ligands with resounding successes in enantioselective catalysis ((a) Wang, F.; Liu, L.-J.; Wang, W.; Li, S.; Shi, M. Chiral NHC-Metal-Based Asymmetric Catalysis. Coord. Chem. Rev. 2012, 256, 804-853. (b) Janssen-Müller, D.; Schlepphorst, C.; Glorius, F. Privileged Chiral N-Heterocyclic Carbene Ligands for Asymmetric Transition-Metal Catalysis. Chem. Soc. Rev., 2017, 46, 4845-4854).

Recently however, a new class of chiral carbenes namely chiral cyclic (alkyl) (amino) carbenes (CAACs) arose as a contender to NHCs dominion over carbene driven enantioselective catalysis ((a) Lavallo, V.; Canac, Y.; Prasang, C.; Donnadieu, B.; Bertrand, G. Angew. Chem., Int. Ed. 2005, 44, 5705-5709. For recent reviews on CAACs, see: (b) Soleilhavoup, M.; Bertrand, G. Acc. Chem. Res. 2015, 48, 256-266; (c) Melaimi, M., Jazzar, R., Soleilhavoup, M., Bertrand, G. Angew. Chem. Int. Ed., 2017, 56, 10056; d) Morvan, J; Mauduit, M; Bertrand, G; Jazzar, R. ACS Catal., 2021, 11, 1714). In recent years, CAAC ligands have been shown by various research groups to afford robust and well-defined CAAC-metal transition complexes. The latter demonstrated that their unique electronic (more sigma-donating and pi-accepting than NHCs) and steric properties allow for the improvement of known catalytic processes (Ru: see for instance: (a) Marx, V. M.; Sullivan, A. H.; Melaimi, M.; Virgil, S. C.; Keitz, B. K.; Weinberger, D. S.; Bertrand, G.; Grubbs, R. H. Angew. Chem., Int. Ed. 2015, 54, 1919. (b) Zhang, J.; Song, S.; Wang, X.; Jiao, J.; Shi, M. Chem. Commun. 2013, 49, 9491. (c) Anderson, D. R.; Lavallo, V.; O'Leary, D. J.; Bertrand, G.; Grubbs, R. H. Angew. Chem., Int. Ed. 2007, 46, 7262; For Pd, see: (a) V. Lavallo, Y. Canac, C. Präsang, B. Donnadieu and G. Bertrand, Angew. Chem., Int. Ed., 2005, 44, 5705; (b) C. M. Weinstein, G. P. Junor, D. R. Tolentino, R. Jazzar, M. Melaimi and G. Bertrand, J. Am. Chem. Soc., 2018, 140, 9255. For Rh, see: (a) M. P. Wiesenfeldt, Z. Nairoukh, W. Li and F. Glorius, Science, 2017, 357, 908; (b) Y. Wei, B. Rao, X. Cong and X. Zeng, J. Am. Chem. Soc., 2015, 137, 9250; (c) Z. Nairoukh, M. Wollenburg, C. Schlepphorst, K. Bergander and F. Glorius, Nat. Chem., 2019, 11, 264) as well as promoting novel reactions with coinage metals (For Cu, see: (a) E. A. Romero, R. Jazzar and G. Bertrand, Chem. Sci., 2017, 8, 165; (b) J. Chu, D. Munz, R. Jazzar, M. Melaimi and G. Bertrand, J. Am. Chem. Soc., 2016, 138, 7884. For Au, see: (a) X. Hu, D. Martin, M. Melaimi and G. Bertrand, J. Am. Chem. Soc., 2014, 136, 13594; (b) R. Kinjo, B. Donnadieu and G. Bertrand, Angew. Chem., Int. Ed., 2011, 50, 5560; (b) L. Jin, D. S. Weinberger, M. Melaimi, C. E. Moore, A. L. Rheingold and G. Bertrand, Angew. Chem., Int. Ed., 2014, 53, 9059).

Surprisingly, as recently noted by Glorius and co-workers (D. Janssen-Mueller, C. Schlepphortst and F. Glorius, Chem. Soc. Rev., 2017, 46, 4845) despite the existence of a variety of stable heterocyclic carbenes, only diaminocarbenes have been intensively used as ligands for enantioselective transformations. Indeed, regarding chiral CAAC ligands, only two applications were reported in the literature ((a) Pichon, D.; Soleilhavoup, M.; Morvan, J.; Junor, G. P.; Vives, T.; Crevisy, C.; Lavallo, V.; Campagne, J.-M.; Mauduit, M.; Jazzar, R.; Bertrand, G. The Debut of Cyclic (Alkyl)(Amino)Carbenes (CAACs) in Enantioselective Catalysis. Chem. Sci. 2019, 10, 7807; (b) Morvan, J.; Vermersch, F.; Zhang, Z.; Falivene, L.; Vives, T.; Dorcet, V.; Roisnel, T.; Crevisy, C.; Cavallo, L.; Vanthuyne, N.; Bertrand, G.; Jazzar, R.; Mauduit, M. Optically Pure C1-Symmetric Cyclic(alkyl)(amino) carbene (CAAC) Ruthenium-Complexes for Asymmetric Olefin Metathesis. J. Am. Chem. Soc. 2020, 142, 19895).

Nevertheless, as a major drawback, these optically pure CAAC ligands were obtained following tedious low yielding procedures, and very often only one of the two enantiomers was prepared.

The aim of the present invention is thus to provide new optically pure enantiomers of iminium salts as precursors of optically pure cyclic (alkyl)(amino) carbenes (CAACs) ligands used for asymmetric catalysis.

Another aim of the present invention is to provide new optically pure enantiomers of iminium salts that could be prepared by a process that does not require any optically pure or enantioenriched raw materials, and, in other words, that does not require the use of chiral chemical compounds.

Another aim of the present invention is to provide a process for the preparation of new optically pure enantiomers of iminium salts that is more economic and faster in comparison with the prior art processes.

Therefore, the present invention relates to a process for the preparation of an optically pure (+) or (−) enantiomer of an iminium salt having the following formula (I):

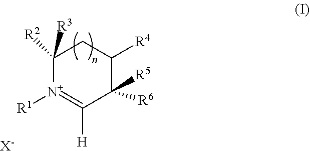

wherein:
   R¹ is a (C₆-C₁₄)aryl group, a (C₁-C₆)alkyl group or a (C₈-C₂₀)cycloalkyl group, said aryl group being optionally substituted with at least one substituent chosen from the group consisting of: halogen, (C₆-C₁₀)aryl group, and (C₁-C₆)alkyl group, said alkyl group being optionally substituted with one or several phenyl group(s);
or R¹ is a —NR′ₐR′_{b} group, R′ₐ and R′_{b} being independently from each other selected from the group consisting of: H, (C₁-C₆)alkyl, and (C₆-C₁₀)aryl, or R′ₐ and R′_{b} form together with the nitrogen atom carrying them a N(CH₂)₂₊ₘ heterocyclyl ring, m being 0 or an integer comprised from 1 to 6;
   R² is H, a (C₆-C₁₀)aryl group or a (C₁-C₆)alkyl group;
   R³ is a (C₁-C₆)alkyl group; or R² and R³ may together form, with the carbon atom carrying them, a (C₃-C₆)cycloalkyl;
   R⁵ is selected from the following groups: (C₆-C₂₀)aryl, (C₁-C₁₀)alkyl, and (C₃-C₁₂)cycloalkyl group,
said alkyl group being optionally substituted with at least one substituent chosen from the (C₆-C₁₀)aryl groups, and
said aryl group being optionally substituted with at least one substituent chosen from the group consisting of: (C₁-C₆)alkyl, optionally substituted with one or several phenyl group(s), (C₆-C₁₀)aryl(C₁-C₆)alkyl, and (C₆-C₁₀)aryl, optionally substituted with one or several substituents, in particular selected in the group consisting of: (C₁-C₆)alkylamino, di(C₁-C₆)alkylamino, (C₁-C₆) alkoxy, and (C₁-C₆)alkyl;
   R⁶ is selected from the following groups: (C₆-C₂₀)aryl, (C₁-C₁₀)alkyl, (C₃-C₁₂)cycloalkyl, heteroaryl, (C₆-C₁₀)aryl(C₁-C₆)alkyl, and heteroaryl(C₁-C₆)alkyl, said aryl group being optionally substituted with at least one substituent chosen from the group consisting of: (C₁-C₆)alkyl, optionally substituted with one or several phenyl group(s), (C₆-C₁₀)aryl(C₁-C₆)alkyl, and (C₆-C₁₀)aryl, optionally substituted with one or several substituents, in particular selected in the group consisting of: (C₁-C₆)alkylamino, di(C₁-C₆)alkylamino, (C₁-C₆) alkoxy, and (C₁-C₆)alkyl;
with the proviso that R⁶ is different from R⁵;
or R⁵ and R⁶, taken together with the carbon atom to which they are attached, form a five-, six-, or ten-membered cycloalkyl or heterocyclyl ring;
   R⁴ is H or a (C₁-C₆)alkyl group;
   n is 0 or an integer comprised between 1 and 3;
or R³ and R⁵, taken together with the carbon atom to which they are attached, form a six-, seven- or eight-membered cycloalkyl ring;
   X⁻ is a counteranion,
said salt being in the form of an optically pure (+) or (−) enantiomer,
said process comprising the following steps:
a) a reduction step of an iminium salt having the following formula (II), said salt being in the form of a racemic mixture:

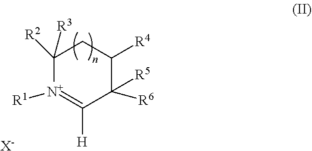

R¹, R², R³, R⁴, R⁵, R⁶, n, and X⁻ are as defined above in formula (I), in order to obtain a compound having the formula (III):

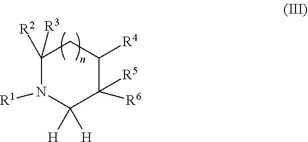

R¹, R², R³, R⁴, R⁵, R⁶, and n are as defined above in formula (I),
said compound of formula (III) being in the form of a racemic mixture,
b) a step of chiral HPLC separation of the compound of formula (III) in the form of a racemic mixture, for obtaining an optically pure (+) or (−) enantiomer compound having the formula (IV):

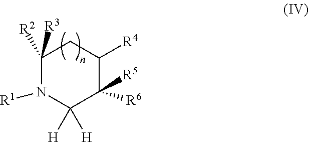

R¹, R², R³, R⁴, R⁵, R⁶, and n are as defined above in formula (I),
said compound of formula (IV) being in the form of an optically pure (+) or (−) enantiomer,
c) an oxidation step of the compound of formula (IV) for obtaining the compound of formula (I),
d) and optionally a counteranion exchange step.

The present invention also relates to an optically pure (+) or (−) enantiomer of an iminium salt having the following formula (I):

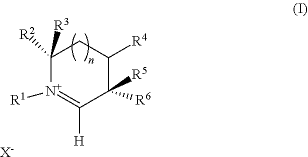

wherein:
   R¹ is a (C₆-C₁₄)aryl group, a (C₁-C₆)alkyl group or a (C₈-C₂₀)cycloalkyl group, said aryl group being optionally substituted with at least one substituent chosen from the group consisting of: halogen, (C₆-C₁₀)aryl group, and (C₁-C₆)alkyl group, said alkyl group being optionally substituted with one or several phenyl group(s);
or R¹ is a —NR′ₐR′_{b} group, R′ₐ and R′_{b} being independently from each other selected from the group consisting of: H, (C₁-C₆)alkyl, and (C₆-C₁₀)aryl, or R′ₐ and R′_{b} form together with the nitrogen atom carrying them a N(CH₂)₂₊ₘ heterocyclyl ring, m being 0 or an integer comprised from 1 to 6;
   R² is H, a (C₆-C₁₀)aryl group or a (C₁-C₆)alkyl group;
   R³ is a (C₁-C₆)alkyl group; or R² and R³ may together form, with the carbon atom carrying them, a (C₃-C₆)cycloalkyl;
   R⁵ is selected from the following groups: (C₆-C₂₀)aryl, (C₁-C₁₀)alkyl, and (C₃-C₁₂)cycloalkyl group,
said alkyl group being optionally substituted with at least one substituent chosen from the (C₆-C₁₀)aryl groups, and
said aryl group being optionally substituted with at least one substituent chosen from the group consisting of: (C₁-C₆)alkyl, optionally substituted with one or several phenyl group(s), (C₆-C₁₀)aryl(C₁-C₆)alkyl, and (C₆-C₁₀)aryl, optionally substituted with one or several substituents, in particular selected in the group consisting of: (C₁-C₆)alkylamino, di(C₁-C₆)alkylamino, (C₁-C₆) alkoxy, and (C₁-C₆)alkyl;
   R⁶ is selected from the following groups: (C₆-C₂₀)aryl, (C₁-C₁₀)alkyl, (C₃-C₁₂)cycloalkyl, heteroaryl, (C₆-C₁₀)aryl(C₁-C₆)alkyl, and heteroaryl(C₁-C₆)alkyl, said aryl group being optionally substituted with at least one substituent chosen from the group consisting of: (C₁-C₆)alkyl, optionally substituted with one or several phenyl group(s), (C₆-C₁₀)aryl(C₁-C₆)alkyl, and (C₆-C₁₀)aryl, optionally substituted with one or several substituents, in particular selected in the group consisting of: (C₁-C₆)alkylamino, di(C₁-C₆)alkylamino, (C₁-C₆) alkoxy, and (C₁-C₆)alkyl;
with the proviso that R⁶ is different from R⁵;
or R⁵ and R⁶, taken together with the carbon atom to which they are attached, form a five-, six-, or ten-membered cycloalkyl or heterocyclyl ring;
   R⁴ is H or a (C₁-C₆)alkyl group;
   n is 0 or an integer comprised between 1 and 3;
or R³ and R⁵, taken together with the carbon atom to which they are attached, form a six-, seven- or eight-membered cycloalkyl ring;
   X⁻ is a counteranion,
said salt being in the form of an optically pure (+) or (−) enantiomer.

A preferred family of optically pure (+) or (−) enantiomers of iminium salt according to the invention consists of salts having the following formula (I-1):

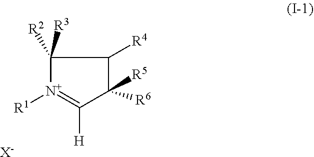

wherein R¹, R², R³, R⁴, R⁵, R⁶ and X⁻ are as defined in formula (I).

Salts of formula (I-1) correspond to salts of formula (I) wherein n=0.

Preferably, in formula (I-1), R⁴ is H.

Thus, the present invention also relates to said salts, and to the process for the preparation of said salts as defined above, starting from compounds having the formula (II) wherein n=0.

A preferred family of optically pure (+) or (−) enantiomers of iminium salt according to the invention consists of salts having the following formula (I) as defined above wherein n is an integer comprised between 1 and 3, and is preferably 1.

A preferred family of optically pure (+) or (−) enantiomers of iminium salt according to the invention consists of salts having the following formula (I-2):

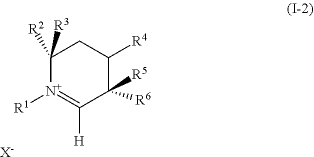

wherein R¹, R², R³, R⁴, R⁵, R⁶ and X⁻ are as defined in formula (I).

Salts of formula (I-2) correspond to salts of formula (I) wherein n=1.

Thus, the present invention also relates to said salts, and to the process for the preparation of said salts as defined above, starting from compounds having the formula (II) wherein n=1.

A preferred family of optically pure (+) or (−) enantiomers of iminium salt according to the invention consists of salts having the following formula (I-3):

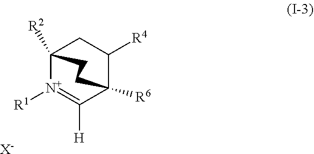

wherein R¹, R², R⁴, R⁶ and X⁻ are as defined in formula (I).

Preferably, in formula (I-3), R² is a (C₁-C₆)alkyl group, such as methyl.

Preferably, in formula (I-3), R⁶ is a (C₁-C₆)alkyl group, such as methyl, ethyl, or propyl groups, optionally substituted by a phenyl group.

Preferably, in formula (I-3), R⁴ is a (C₁-C₆)alkyl group.

According to an embodiment, in formulae (I), (I-1), (I-2), and (I-3), R¹ is a substituted phenyl group. Preferably, in formulae (I), (I-1), (I-2), and (I-3), R¹ is a phenyl group being substituted with at least one or two substituent(s), said substituents being selected from the (C₁-C₆)alkyl groups, such as methyl, ethyl or isopropyl.

Preferably, in formulae (I), (I-1), (I-2), and (I-3), R¹ is a phenyl group being substituted with two substituent(s) in ortho position, said substituents being identical or different.

Preferably, in formulae (I), (I-1), (I-2), and (I-3), R¹ is a phenyl group being substituted with two substituents in ortho position, said substituents being identical or different, said substituents being preferably (C₁-C₆)alkyl groups, such as methyl, ethyl or isopropyl.

As preferred R¹ groups, one may cite phenyl groups with two alkyl groups, in particular two identical alkyl groups, such as ethyl or isopropyl, in ortho position.

According to an embodiment, in formula (I) or also in formulae (I-1), (I-2) or (I-3), R¹ is a (C₆-C₁₀)aryl group substituted with at least one substituent chosen from the (C₁-C₆)alkyl groups, preferably a phenyl group substituted with two alkyl groups, such as methyl, isopropyl or ethyl groups, and/or wherein R² is a (C₁-C₆)alkyl group such as a methyl group.

According to an embodiment, in formula (I) or also in formulae (I-1), (I-2) or (I-3), R² and R³ are identical, and are preferably a methyl group.

According to an embodiment, in formula (I) or also in formulae (I-1), (I-2) or (I-3), R² and R³ are different, R² being preferably a (C₁-C₆)alkyl group such as a methyl group and R³ being preferably H or a (C₆-C₁₀)aryl group such as a phenyl group.

According to an embodiment, in formula (I) or also in formulae (I-1), (I-2) or (I-3), R⁴ is H.

According to an embodiment, in formula (I) or also in formulae (I-1), (I-2) or (I-3), R⁶ is an alkyl group as defined above, preferably a methyl group, and R⁵ is an aryl group as defined above. Preferably, said aryl group is a (C₆-C₁₀)aryl group, such as a naphthyl group or a phenyl group, being substituted with at least one, in particular one, two or three, substituent(s), said substituent(s) being selected from the group consisting of: (C₁-C₆)alkyl groups, such as methyl or isopropyl, (C₁-C₆)alkylamino groups, di(C₁-C₆)alkylamino groups, (C₁-C₆)alkoxy groups, (C₆-C₁₀)aryl groups such as phenyl, and —CH(Ar)₂, such as —CH(Ph)₂, Ar being an aryl group. Preferably, R⁵ is a phenyl group being substituted with two substituents in meta position, said substituents being identical or different, preferably identical, such as methyl, isopropyl or tertiobutyl.

According to an embodiment, in formula (I) or also in formulae (I-1), (I-2) or (I-3), R⁵ is a cyclohexyl group.

According to an embodiment, in formula (I) or also in formulae (I-1), (I-2) or (I-3), R⁴ is an aryl group as defined above and R⁵ is an alkyl group as defined above, preferably a methyl group. Preferably, said aryl group is a naphthyl radical or a (C₆-C₁₀)aryl group, such as a phenyl group, being substituted with at least one, in particular one, two or three, substituent(s), said substituent(s) being selected from the group consisting of: (C₁-C₆)alkyl groups, such as methyl or isopropyl, (C₁-C₆)alkylamino groups, di(C₁-C₆)alkylamino groups, (C₁-C₆)alkoxy groups, (C₆-C₁₀)aryl groups such as phenyl, and —CH(Ar)₂, such as —CH(Ph)₂, Ar being an aryl group. Preferably, R⁶ is a phenyl group being substituted with two substituents in meta position, said substituents being identical or different, preferably identical, such as methyl, isopropyl or tertiobutyl.

As preferred aryl groups for R⁶ (or R⁵), the followings may be mentioned:

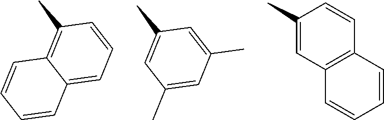

According to an embodiment, in formula (I) or also in formulae (I-1), (I-2) or (I-3), R⁶ is an aryl group as defined above, said aryl group being optionally substituted with at least one substituent as defined hereafter, and R⁵ is an alkyl group as defined above, preferably a methyl group.

According to an embodiment, in formula (I) or also in formulae (I-1), (I-2) or (I-3), R⁵ is an aryl group as defined above, said aryl group being optionally substituted with at least one substituent as defined hereafter, and R⁶ is an alkyl group as defined above, preferably a methyl group.

According to an embodiment, in formula (I) or also in formulae (I-1), (I-2) or (I-3), R⁶ is an alkyl group as defined above, preferably a methyl group, and R⁵ is selected from the group consisting of: (C₁-C₁₀)alkyl, such as tertio-butyl group, and (C₃-C₁₂)cycloalkyl groups.

According to an embodiment, in formula (I) or also in formulae (I-1), (I-2) or (I-3), R⁵ is an alkyl group as defined above, preferably a methyl group, and R⁶ is selected from the group consisting of: (C₁-C₁₀)alkyl, such as tertio-butyl group, and (C₃-C₁₂)cycloalkyl groups.

As preferred cycloakyl groups for R⁺ (or R⁵), the followings may be mentioned:

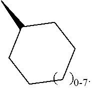

According to an embodiment, in formula (I) or also in formulae (I-1), (I-2) or (I-3), R⁶ is an alkyl group as defined above, preferably a methyl group, and R⁵ is an aryl group as defined above, preferably a phenyl group.

According to an embodiment, in formula (I) or also in formulae (I-1), (I-2) or (I-3), R⁵ is an alkyl group as defined above, preferably a methyl group, and R⁶ is an aryl group as defined above, preferably a phenyl group.

According to an embodiment, in formula (I) or also in formulae (I-1), (I-2) or (I-3), R⁵ and R⁶ are different and selected from the following groups: (C₆-C₁₀)aryl such as phenyl or naphthyl, (C₁-C₆)alkyl such as methyl, and (C₃-C₆)cycloalkyl such as cyclohexyl, said aryl group being optionally substituted with two substituents selected from the (C₁-C₆)alkyl groups.

According to an embodiment, in formula (I) or also in formulae (I-1), (I-2) or (I-3), X⁻ is a counteranion, preferably selected from the group consisting of: BF₄⁻, I⁻, Cl⁻, OTf⁻, Br⁻, PF₆⁻, SbF₆⁻, and B(Ar)₄⁻, Ar representing an aryl group, such as BPh₄⁻. As counteranions the followings may also be mentioned: MXₙ⁻ e.g. CuCl₂⁻, AuBr₂⁻, [Pd(η3-cin)Cl₂]⁻, FeCl₄⁻ (see Ekaterina A. Martynova, Nikolaos V. Tzouras, Gianmarco Pisanò, Catherine S. J. Cazin and Steven P. Nolan (Chemical Communications, 32, 2021)) or [NiCl₄²⁻] (see Mickaël Henrion, Sonia Duarte Barroso, Ana M. Martins, Vincent Ritleng, Michael J. Chetcuti (Polyhedron, volume 87, February 2015, p. 398-402) or Yan-Chao Xu, Jie Zhang, Hong-Mei Sun, Qi Shen and Yong Zhang (Dalton Transactions, 23, 2013)).

Any counteranion known from the skilled person may be used. Other examples may be found for example Han Vinh Huynh, Truc Tien Lam and Huyen T. T. Luong (RSC Advances, issue 61, 2018).

According to a preferred embodiment, X⁻ is BF₄⁻.

In the context of the present invention, the expression “Cₜ-C_{z}” means a carbon-based chain which can have from t to z carbon atoms, for example C₁-C₃ means a carbon-based chain which can have from 1 to 3 carbon atoms.

According to the invention, the term “halogen” means: a fluorine, a chlorine, a bromine or an iodine.

According to the invention, the term “alkyl group” means: a linear or branched, saturated, hydrocarbon-based aliphatic group comprising, unless otherwise mentioned, from 1 to 12 carbon atoms. By way of examples, mention may be made of methyl, ethyl, n-propyl, isopropyl, butyl, isobutyl, tert-butyl or pentyl groups;

According to the invention, the term “cycloalkyl group” means: a cyclic carbon-based group comprising, unless otherwise mentioned, from 3 to 12 carbon atoms. By way of examples, mention may be made of cyclopropyl, cyclobutyl, cyclopentyl, cyclohexyl, or adamantyl etc. groups;

According to the invention, the term “alkoxy group” means: an —O-alkyl radical where the alkyl group is as previously defined. By way of examples, mention may be made of —O—(C₁-C₄)alkyl groups, and in particular the —O-methyl group, the —O-ethyl group as —O—C₃alkyl group, the —O-propyl group, the —O-isopropyl group, and as —O—C₄alkyl group, the —O-butyl, —O-isobutyl or —O-tert-butyl group.

According to the invention, the term “aryl group” means: a cyclic aromatic group comprising between 6 and 10 carbon atoms. By way of examples of aryl groups, mention may be made of phenyl or naphthyl groups.

According to the invention, the term “heteroaryl” means: a 5- to 10-membered aromatic monocyclic or bicyclic group containing from 1 to 4 heteroatoms selected from O, S or N. By way of examples, mention may be made of imidazolyl, thiazolyl, oxazolyl, furanyl, thiophenyl, pyrazolyl, oxadiazolyl, tetrazolyl, pyridinyl, pyrazinyl, pyrimidinyl, pyridazinyl, indolyl, benzofuranyl, benzothiophenyl, benzoxazolyl, benzimidazolyl, indazolyl, benzothiazolyl, isobenzothiazolyl, benzotriazolyl, quinolinyl and isoquinolinyl groups.

By way of a heteroaryl comprising 5 to 6 atoms, including 1 to 4 nitrogen atoms, mention may in particular be made of the following representative groups: pyrrolyl, pyrazolyl, 1,2,3-triazolyl, 1,2,4-triazolyl, tetrazolyl and 1,2,3-triazinyl.

Mention may also be made, by way of heteroaryl, of thiophenyl, oxazolyl, furazanyl, 1,2,4-thiadiazolyl, naphthyridinyl, quinoxalinyl, phthalazinyl, imidazo[1,2-a]pyridine, imidazo[2,1-b]thiazolyl, cinnolinyl, benzofurazanyl, azaindolyl, benzimidazolyl, benzothiophenyl, thienopyridyl, thienopyrimidinyl, pyrrolopyridyl, imidazopyridyl, benzoazaindole, 1,2,4-triazinyl, indolizinyl, isoxazolyl, isoquinolinyl, isothiazolyl, purinyl, quinazolinyl, quinolinyl, isoquinolyl, 1,3,4-thiadiazolyl, thiazolyl, isothiazolyl, carbazolyl, and also the corresponding groups resulting from their fusion or from fusion with the phenyl nucleus.

According to the invention, the term “heterocycloalkyl” means: a 4- to 10-membered, saturated or partially unsaturated, monocyclic or bicyclic group comprising from one to three heteroatoms selected from O, S or N; the heterocycloalkyl group may be attached to the rest of the molecule via a carbon atom or via a heteroatom; the term bicyclic heterocycloalkyl includes fused bicycles and spiro-type rings.

By way of saturated heterocycloalkyl comprising from 5 to 6 atoms, mention may be made of oxetanyl, tetrahydrofuranyl, dioxolanyl, pyrrolidinyl, azepinyl, oxazepinyl, pyrazolidinyl, imidazolidinyl, tetrahydrothiophenyl, dithiolanyl, thiazolidinyl, tetrahydropyranyl, tetrahydropyridinyl, dioxanyl, morpholinyl, piperidinyl, piperazinyl, tetrahydrothiopyranyl, dithianyl, thiomorpholinyl or isoxazolidinyl.

When the heterocycloalkyl is substituted, the substitution(s) may be on one (or more) carbon atom(s) and/or on the heteroatom(s). When the heterocycloalkyl comprises several substituents, they may be borne by one and the same atom or different atoms.

The abovementioned “alkyl”, “cycloalkyl”, “aryl”, “heteroaryl” and “heterocycloalkyl” radicals can be substituted with one or more substituents. Among these substituents, mention may be made of the following groups: amino, hydroxyl, thiol, oxo, halogen, alkyl, alkoxy, alkylthio, alkylamino, aryloxy, arylalkoxy, cyano, trifluoromethyl, carboxy or carboxyalkyl.

According to the invention, the term “alkylthio” means: an —S-alkyl group, the alkyl group being as defined above.

According to the invention, the term “arylthio” means: an —S-aryl group, the aryl group being as defined above.

According to the invention, the term “alkylamino” means: an —NH-alkyl group, the alkyl group being as defined above.

According to the invention, the term “cycloalkyloxy” means: an —O-cycloalkyl group, the cycloalkyl group being as defined above.

According to the invention, the term “aryloxy” means: an —O-aryl group, the aryl group being as defined above.

According to the invention, the term “(hetero)arylalkoxy” means: a (hetero)aryl-alkoxy-group, the (hetero)aryl and alkoxy groups being as defined above.

According to the invention, the term “alkylcarbonyl” means a —CO-alkyl group, the alkyl group being as defined above.

According to the invention, the term “alkoxylcarbonyl” means a —CO—O-alkyl group, the alkyl group being as defined above.

According to the invention, the term “arylcarbonyl” means a —CO-aryl group, the aryl group being as defined above.

According to the invention, the term “aryloxycarbonyl” means a —CO-aryloxy group, the aryloxy group being as defined above.

According to the invention, the term “alkylsulfonyl” means a —SO₂-alkyl group, the alkyl group being as defined above.

According to the invention, the term “arylsulfonyl” means a —SO₂-aryl group, the aryl group being as defined above.

According to the invention, the term “alkylsulfinyl” means a —SO-alkyl group, the alkyl group being as defined above.

According to the invention, the term “arylsulfinyl” means a —SO-aryl group, the aryl group being as defined above.

According to the invention, the term “carboxyalkyl” means: an HOOC-alkyl-group, the alkyl group being as defined above. As examples of carboxyalkyl groups, mention may in particular be made of carboxymethyl or carboxyethyl.

According to the invention, the term “carboxyl” means: a COOH group.

According to the invention, the term “oxo” means: “═O”.

When an alkyl radical is substituted with an aryl group, the term “arylalkyl” or “aralkyl” radical is used. The “arylalkyl” or “aralkyl” radicals are aryl-alkyl-radicals, the aryl and alkyl groups being as defined above. Among the arylalkyl radicals, mention may in particular be made of the benzyl or phenethyl radicals.

As preferred salts according to the invention, the followings may be mentioned:

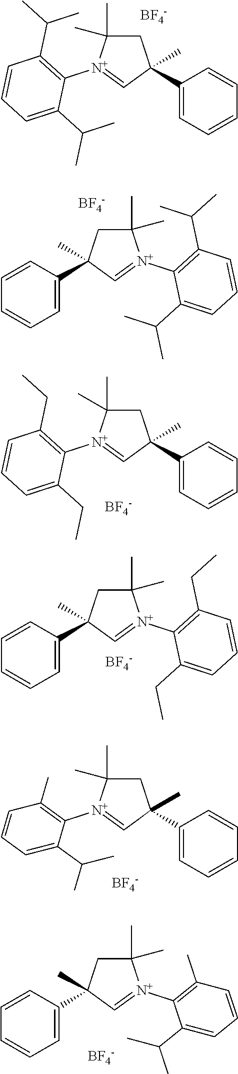

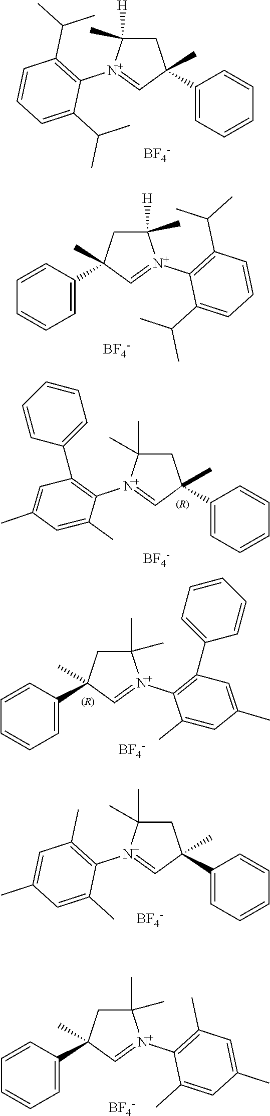

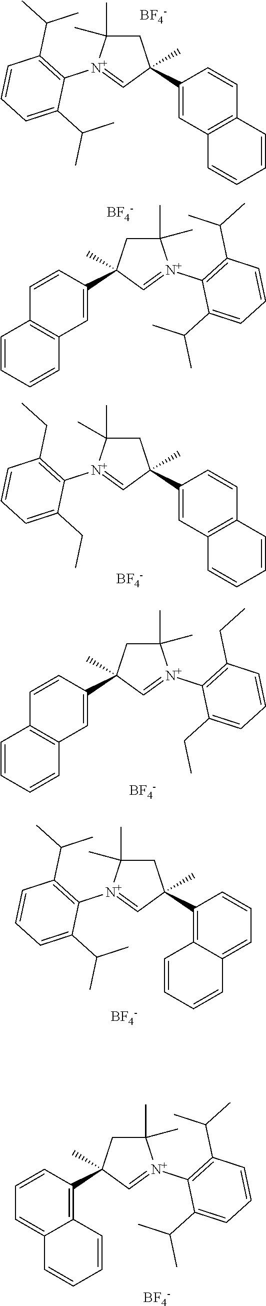

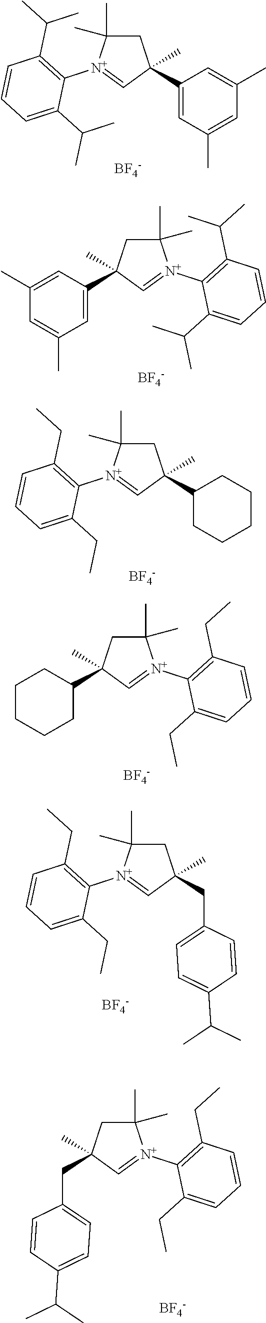

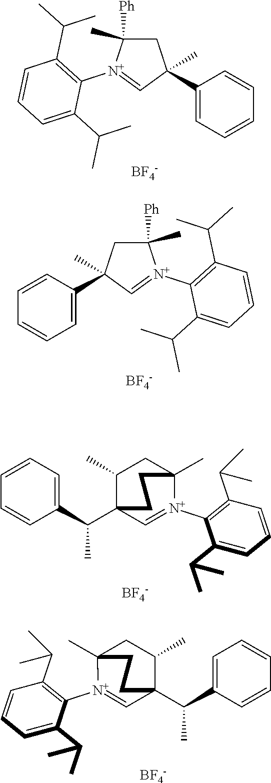

As preferred salts according to the invention, the followings may be mentioned:

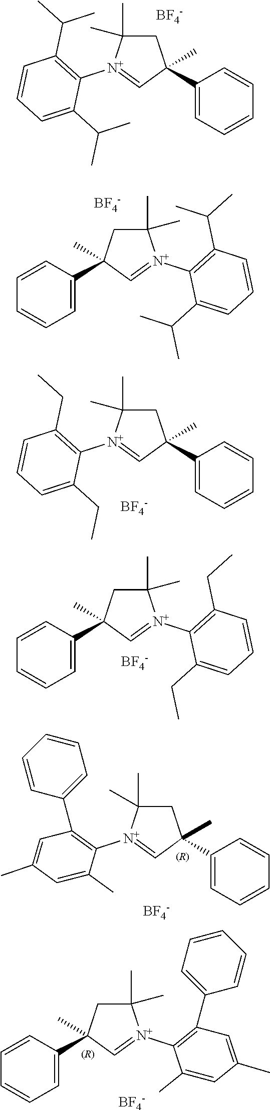

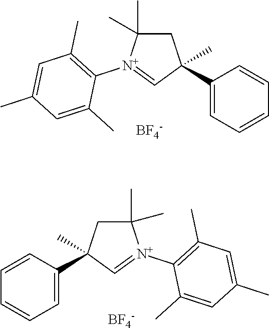

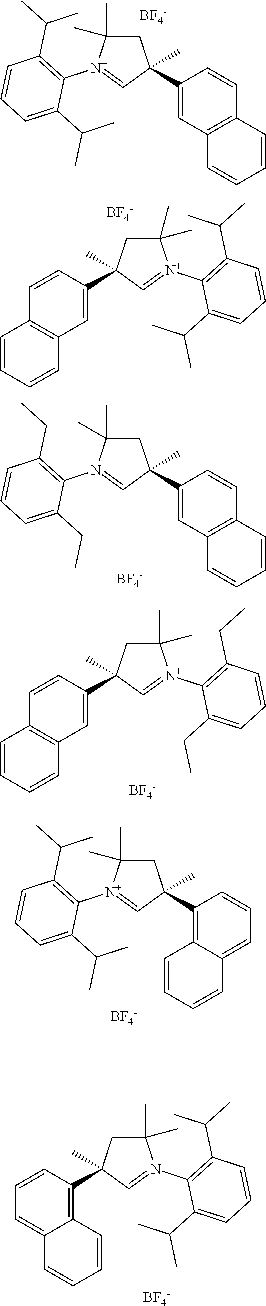

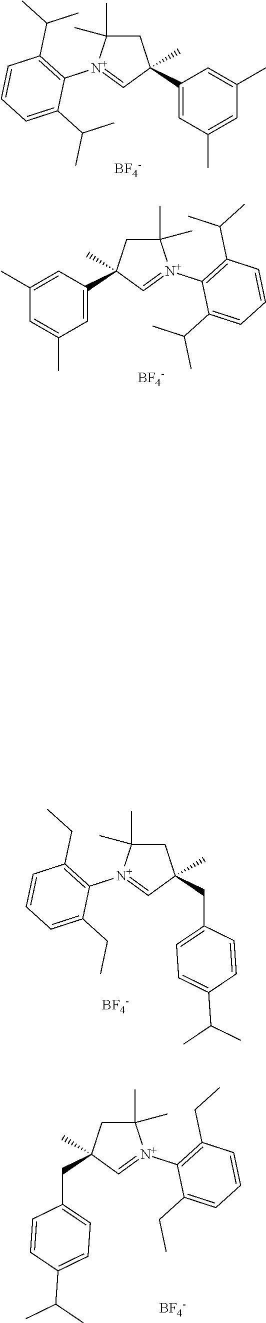

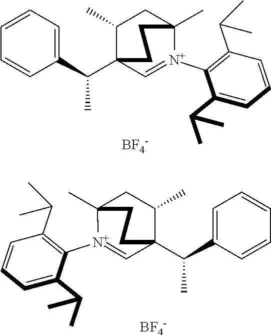

As mentioned above, the process according to the invention comprises a first step consisting in a reduction step of an iminium salt of formula (II), said salt being in the form of a racemic mixture, and said step giving a compound of formula (III) also in the form of a racemic mixture.

Formulae (II) and (III) are as defined above. In these formulae, n, R¹, R², R³, R⁴, R⁵, and R⁶ are as defined above in formulae (I), (I-1), (I-2) or (I-3).

The reduction step of the process of the invention is carried out in particular with a reduction agent. Concerning the reduction agent, see Reductions by the Alumino- and Borohydrides in Organic Synthesis, 2nd edition (Seyden-Penne, Jacqueline), Chapter 3.3.1 Imines and Iminium Salts page 122, or also Hitchhiker's Guide to Reductive Amination (Evgeniya Podyacheva, Oleg I. Afanasyev, Alexey A. Tsygankov, Maria Makarova, Denis Chusov) in Synthesis 2019; 51 (13): 2667-2677).

According to an embodiment, the reduction agent is selected from the group consisting of: LiAlH₄, NaBH₄, diisobutylaluminium hydride (DIBAL), lithium triethylborohydride (LITEBH), sodium bis(2-methoxyethoxy)aluminium hydride (Red-Al), and cyanoborohydrides.

Preferably, the reduction step is carried out in a solvent, said solvent being in particular THF. According to an embodiment, the reduction step is carried out at a temperature comprised between 0° C. and room temperature (20-24° C.).

According to an embodiment, the reduction step may be carried out with a HPLC column.

The process of the invention also comprises a step of chiral HPLC separation of the compound of formula (III) in the form of a racemic mixture, for obtaining an optically pure (+) or (−) enantiomer compound of formula (IV), said compound of formula (IV) being in the form of an optically pure (+) or (−) enantiomer.

Formula (IV) is as defined above. In this formula, n, R¹, R², R³, R⁴, R⁵, and R⁶ are as defined above in formulae (I), (I-1), (I-2) or (I-3).

Preferably, said step of chiral HPLC separation is carried out with a HPLC column comprising cellulose, in particular substituted with chloro-phenylcarbamate as chiral stationary phase.

As HPLC columns, the Lux® columns may be mentioned such as Lux-Cellulose-3 (Cellulose tris(4-methylbenzoate)) or Lux-Cellulose-4 (Cellulose tris(4-chloro-3-methylphenylcarbamate)).

According to an embodiment, ethanol or heptane is used as mobile phase.

The process of the invention also comprises an oxidation step of the compound of formula (IV) for obtaining the salt of formula (I), said salt of formula (I) being in the form of an optically pure (+) or (−) enantiomer.

The oxidation step of the invention is in particular carried out with an oxidation agent. According to an embodiment, the oxidation agent is selected from the group consisting of: Br₂, N-bromosuccinimide, I₂, N-iodosuccinimide, a copper (II) compound in particular CuX₂ (X being for example Cl, Br, I or OTf), and more particularly CuCl₂, Cl₂, a hypervalent iodine compound such as 2-iodoxybenzoic acid (IBX), 2,3-dichloro-5,6-dicyano-1,4-benzoquinone (DDQ), tetra-N-butylammonium iodide (TBAI), and tert-butyl hydroperoxide (TBHP).

Other examples of oxidant agents may be found in “Organocatalysis in Inert C—H Bond Functionalization” of Yan Qin, Lihui Zhu, and Sanzhong Luo (Chem. Rev. 2017, 117, 13, 9433-9520).

Preferably, the oxidation step is carried out in a solvent, said solvent being in particular dichloromethane. According to an embodiment, the oxidation step is carried out at a temperature comprised between 0° C. and room temperature (20-24° C.).

The process of the invention may also comprise a further step consisting in a counteranion exchange.

Such step is carried out by means well-known from the skilled person. It allows for example to obtain the salts of formula (I) wherein X is BF₄ or PF₆. See for example “Anion influences on reactivity and NMR spectroscopic features of NHC precursors” of Han Vinh Huynh, Truc Tien Lam and Huyen T. T. Luong (RSC Advances, issue 61, 2018) or see Ekaterina A. Martynova, Nikolaos V. Tzouras, Gianmarco Pisanò, Catherine S. J. Cazin and Steven P. Nolan (Chemical Communications, 32, 2021).

As methods for this step, the followings may be mentioned:
extraction with saturated solution of salts, or
use of resins, or
use of silver salts (e.g. AgNO₃) for salt metathesis with use of insolubility of silver.

The present invention also relates to the use of the compound of formula (I), or formulae (I-1), (I-2) or (I-3), as defined above as a catalyst, preferably as a catalyst in asymmetric olefin metathesis, optionally in combination with a transition metal.

The present invention also relates to the use of the compound of formula (I), or formulae (I-1), (I-2) or (I-3), as defined above as a catalyst.

The present invention also relates to the use of the compound of formula (I), or formulae (I-1), (I-2) or (I-3), as defined above as a catalyst, in combination with a transition metal other than ruthenium.

The present invention also relates to the use of the compound of formula (I), or formulae (I-1), (I-2) or (I-3), as defined above as a catalyst, in combination with a transition metal selected from the group consisting of: gold, copper, and rhodium.

The present invention also relates to the use of the compound of formula (I), or formulae (I-1), (I-2) or (I-3), as defined above, in combination with a transition metal, in an organic light-emitting diode.

The present invention also relates to the use of the compound of formula (I), or formulae (I-1), (I-2) or (I-3), as defined above, in combination with a transition metal, in an organic light-emitting diode, wherein the transition metal is selected from the group consisting of: gold, copper, and rhodium.

The present invention also relates to an organic light emitting device (OLED) comprising: an anode; a cathode; and an organic layer disposed between the anode and the cathode, wherein the organic layer comprises a compound of formula (I) as defined above, in combination with a transition metal selected from the group consisting of Ru, Os, Ir, Pd, Pt, Cu, Ag, and Au, and can be bonded to other ligands.

### EXAMPLES

### Preparation of the Compounds of the Invention

### General Information

All reactions and subsequent manipulations were performed under an argon atmosphere in an MBraun glovebox or using standard Schlenk techniques, if not stated otherwise. ¹H and ¹³C{¹H} NMR spectra were recorded on a Varian 400 or Bruker Avance 400 at 25° C. ¹H NMR chemical shifts are reported relative to TMS (0 in ppm) and were referenced via residual proton resonances of the corresponding deuterated solvent (CHCl₃: 7.26 ppm; C₆D₅H: 7.16 ppm) whereas ¹³C{¹H} NMR spectra are reported relative to TMS using the natural-abundance carbon resonances (CDCl₃: 77.16 ppm; C₆D₆: 128.0 ppm). Coupling constants are given in Hertz.

### CAAC Salt Reduction to Form H₂ Adducts (Corresponding to Step a) of the Process According to the Invention for the Preparation of Compounds of Formula (III) According to the Invention)

General procedure: In a Schlenk tube under argon, lithium aluminum hydride (2 equiv) was slowly added to a solution of iminium salt (1.0 equiv) in a THF at 0° C. and received suspension was further stirred at room temperature overnight. Reaction mixture was then quenched with mixture of hydrated MgSO₄ and silica and then passed through a short pad of silica which was further washed with Et₂O. Evaporation of the combined organic fractions gives desired CAAC-H₂ adducts as white sticky solids in typical yield of 90%.

### Analytical Data:

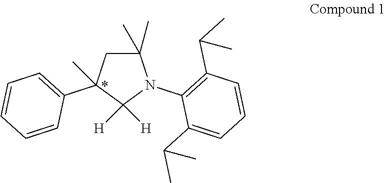 [SMILES: CC(C)c1cccc(C(C)C)c1N1CC(C)(c2ccccc2)CC1(C)C]

¹H NMR (400 MHZ, 25° C., CDCl₃): δ=7.41-7.31 (m, 4H), 7.28-7.20 (m, 2H), 7.16 (ddd, J=17.0, 7.5, 2.0 Hz, 2H), 4.01 (d, J=8.4 Hz, 1H), 3.91 (p, J=6.9 Hz, 1H), 3.50 (d, J=8.6 Hz, 1H), 3.37 (p, J=6.8 Hz, 1H), 2.54 (d, J=12.7 Hz, 1H), 2.30 (dd, J=12.7, 0.8 Hz, 1H), 1.63 (s, 3H), 1.30 (d, J=6.9 Hz, 3H), 1.26 (s, 3H), 1.16 (t, J=6.8 Hz, 6H), 1.08 (d, J=6.8 Hz, 3H), 1.05 (s, 3H).

¹³C{¹H} NMR (100 MHZ, 25° C., CDCl₃): δ=152.3, 152.3, 150.7, 138.4, 128.2, 126.5, 126.0, 125.6, 124.1, 123.8, 65.8, 62.7, 54.4, 45.1, 32.2, 29.7, 29.4, 28.4, 28.2, 26.7, 26.6, 23.1, 22.8.

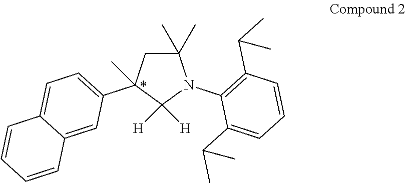 [SMILES: CC(C)c1cccc(C(C)C)c1N1CC(C)(c2ccc3ccccc3c2)CC1(C)C]

¹H NMR (400 MHZ, 25° C., CDCl₃): δ=7.91-7.82 (m, 3H), 7.80 (d, J=1.9 Hz, 1H), 7.56-7.44 (m, 3H), 7.30-7.23 (m, 1H), 7.22 (dd, J=7.7, 2.1 Hz, 1H), 7.17 (dd, J=7.3, 2.1 Hz, 1H), 4.14 (d, J=8.4 Hz, 1H), 3.94 (hept, J=6.9 Hz, 1H), 3.63 (d, J=8.4 Hz, 1H), 3.42 (hept, J=6.8 Hz, 1H), 2.71 (d, J=12.7 Hz, 1H), 2.40 (d, J=12.7 Hz, 1H), 1.73 (s, 3H), 1.35 (d, J=6.9 Hz, 3H), 1.31 (s, 3H), 1.21 (d, J=6.9 Hz, 3H), 1.17 (d, J=6.8 Hz, 3H), 1.11 (d, J=6.8 Hz, 3H), 1.08 (s, 3H).

¹³C{¹H} NMR (100 MHZ, 25° C., CDCl₃): δ=152.3, 152.3, 147.9, 138.5, 133.4, 131.8, 127.9, 127.9, 127.5, 126.6, 125.9, 125.5, 125.3, 124.1, 123.8, 123.6, 65.9, 62.9, 54.6, 45.3, 32.0, 29.7, 29.5, 28.4, 28.3, 26.8, 26.7, 23.2, 22.9.

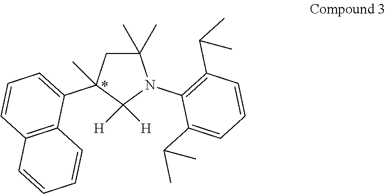 [SMILES: CC(C)c1cccc(C(C)C)c1N1CC(C)(c2cccc3ccccc23)CC1(C)C]

¹H NMR (400 MHZ, 25° C., CDCl₃): δ=8.27-8.18 (m, 1H), 7.96-7.87 (m, 1H), 7.82-7.72 (m, 1H), 7.53-7.42 (m, 4H), 7.29-7.19 (m, 2H), 7.15 (dd, J=7.0, 2.5 Hz, 1H), 4.34 (d, J=8.6 Hz, 1H), 4.06 (hept, J=6.9 Hz, 1H), 3.85 (d, J=8.6 Hz, 1H), 3.34 (hept, J=6.9 Hz, 1H), 2.84 (d, J=12.6 Hz, 1H), 2.67 (d, J=12.5 Hz, 1H), 1.94 (s, 3H), 1.39-1.32 (m, 6H), 1.23 (d, J=6.8 Hz, 3H), 1.17 (d, J=6.8 Hz, 3H), 1.08 (s, 3H), 1.00 (d, J=6.8 Hz, 3H).

¹³C{¹H} NMR (100 MHZ, 25° C., CDCl₃): δ=0.2, 152.1, 146.6, 138.7, 134.9, 131.5, 129.5, 127.2, 126.6, 126.2, 125.2, 125.0, 124.9, 124.2, 123.8, 123.6, 67.2, 62.4, 56.1, 46.0, 31.2, 29.7, 28.8, 28.6, 28.2, 26.7, 26.3, 23.3, 22.9.

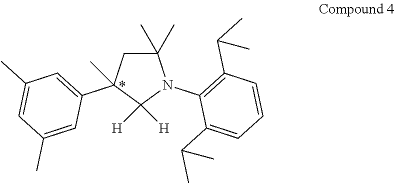 [SMILES: Cc1cc(C)cc(C2(C)CN(c3c(C(C)C)cccc3C(C)C)C(C)(C)C2)c1]

¹H NMR (400 MHZ, 25° C., CDCl₃): δ=7.28-7.14 (m, 3H), 6.99-6.94 (m, 2H), 6.91 (qd, J=1.6, 0.9 Hz, 1H), 4.01 (d, J=8.3 Hz, 1H), 3.99-3.91 (m, 1H), 3.49 (d, J=8.3 Hz, 1H), 3.40 (hept, J=6.8 Hz, 1H), 2.55 (d, J=12.7 Hz, 1H), 2.37 (s, 6H), 2.30 (d, J=12.6 Hz, 1H), 1.64 (s, 3H), 1.32 (d, J=6.9 Hz, 3H), 1.28 (s, 3H), 1.19 (dd, J=12.0, 6.8 Hz, 6H), 1.12 (d, J=6.8 Hz, 3H), 1.08 (s, 3H).

¹³C{¹H} NMR (100 MHZ, 25° C., CDCl₃): δ=152.3, 152.3, 150.9, 138.5, 137.6, 127.3, 126.5, 124.1, 123.8, 123.7, 65.9, 62.7, 54.3, 44.9, 32.3, 29.8, 29.5, 28.5, 28.1, 26.8, 26.6, 23.2, 22.8, 21.6.

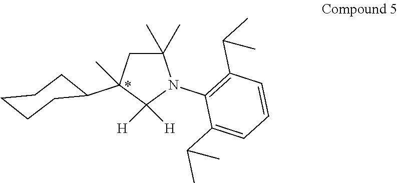 [SMILES: CC(C)c1cccc(C(C)C)c1N1CC(C)(C2CCCCC2)CC1(C)C]

¹H NMR (400 MHZ, 25° C., CDCl₃): δ=7.25-7.18 (m, 1H), 7.18-7.12 (m, 2H), 3.88 (hept, J=6.9 Hz, 1H), 3.56-3.41 (m, 2H), 3.01 (d, J=8.3 Hz, 1H), 1.91 (d, J=12.7 Hz, 1H), 1.86-1.67 (m, 5H), 1.54-1.46 (m, 1H), 1.36-1.25 (m, 10H), 1.22 (s, 4H), 1.20 (s, 4H), 1.17 (d, J=6.8 Hz, 4H), 1.09 (d, J=6.7 Hz, 7H).

¹³C{¹H} NMR (100 MHZ, 25° C., CDCl₃): δ=δ 152.4, 152.1, 138.9, 126.3, 123.9, 123.7, 66.6, 62.6, 55.2, 49.9, 43.5, 29.7, 29.2, 28.8, 28.7, 28.5, 27.9, 27.2, 27.1, 26.9, 26.6, 26.6, 23.2, 22.9, 22.1.

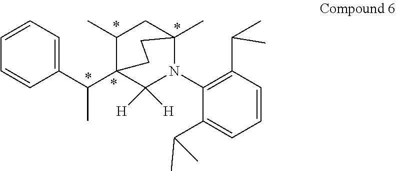 [SMILES: CC(C)c1cccc(C(C)C)c1N1CC2(C(C)c3ccccc3)CCC1(C)CC2C]

Diastereoisomeric ratio of starting iminium salt range between 90/10 to 75/25 and are the same in the product.

### Analytical Data are Given for a Major Dia

¹H NMR (400 MHZ, 25° C., CDCl₃): δ=7.25-7.19 (m, 2H), 7.18-7.12 (m, 2H), 7.09 (m, 3H), 7.00 (dd, J=7.5, 2.0 Hz, 1H), 3.67 (hept, J=6.9 Hz, 1H), 3.21 (dd, J=8.8, 2.8 Hz, 1H), 2.99 (hept, J=6.8 Hz, 1H), 2.79 (q, J=7.3 Hz, 1H), 2.50 (dd, J=8.7, 1.7 Hz, 1H), 2.30-2.15 (m, 1H), 2.05 (dd, J=12.6, 10.9 Hz, 1H), 1.92-1.83 (m, 2H), 1.81-1.71 (m, 2H), 1.75-1.56 (m, 1H), 1.30 (d, J=7.2 Hz, 3H), 1.28-1.21 (m, 6H, overlapping signals), 1.09 (d, J=6.9 Hz, 3H), 0.64 (s, 3H), 0.63 (d, J=6.8 Hz, 3H).

¹³C{¹H} NMR (100 MHZ, 25° C., CDCl₃): δ=151.8, 151.5, 143.9, 143.0, 129.4, 127.6, 126.2, 126.1, 124.1, 123.5, 55.7, 51.3, 47.9, 42.5, 38.7, 35.5, 31.2, 28.5, 27.6, 26.4, 25.7, 24.8, 24.6, 24.5, 23.6, 17.3, 14.1.

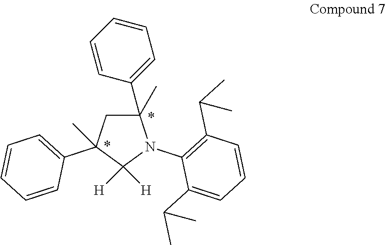 [SMILES: CC(C)c1cccc(C(C)C)c1N1CC(C)(c2ccccc2)CC1(C)c1ccccc1]

Diastereoisomeric ratio: 75/25

¹H NMR (400 MHZ, 25° C., CDCl₃): δ=7.54-7.47 (m, 2H), 7.47-7.35 (m, 3H), 7.33-7.24 (m, 2H), 7.24-7.07 (m, 7H), 6.99-6.87 (m, 2H), 4.23 (p, J=6.9 Hz, OH), 4.14 (d, J=9.2 Hz, 1H), 4.10 (d, J=8.6 Hz, OH), 3.67-3.42 (m, 2H), 3.15 (d, J=12.8 Hz, 1H), 2.82 (td, J=13.2, 6.2 Hz, 2H), 2.47 (dd, J=13.1, 0.8 Hz, OH), 2.16 (p, J=6.8 Hz, OH), 1.80 (s, 1H), 1.73 (s, 1H), 1.56 (s, 2H), 1.44 (d, J=7.0 Hz, 1H), 1.32-1.24 (m, 5H), 1.24-1.18 (m, 1H), 1.14 (d, J=6.8 Hz, 3H), 1.00 (d, J=6.8 Hz, 2H), 0.85 (d, J=6.7 Hz, 1H), 0.30 (d, J=6.8 Hz, 2H), 0.16 (d, J=6.7 Hz, 1H).

¹³C{¹H} NMR (100 MHZ, 25° C., CDCl₃): δ=152.3, 151.9, 151.2, 150.8, 150.1, 146.2, 146.0, 140.1, 138.8, 128.5, 128.3, 127.8, 127.6, 126.7, 126.7, 126.6, 126.1, 126.0, 125.9, 125.8, 125.7, 124.2, 123.9, 123.7, 123.6, 68.0, 67.9, 65.9, 52.0, 45.3, 33.8, 32.9, 30.6, 29.2, 28.8, 28.2, 28.0, 26.8, 26.8, 26.7, 26.2, 25.6, 23.7, 23.0, 21.5, 21.1.

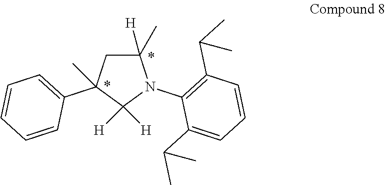 [SMILES: CC(C)c1cccc(C(C)C)c1N1CC(C)(c2ccccc2)CC1C]

Diastereoisomeric ratio: 55/45

¹H NMR (400 MHZ, 25° C., CDCl₃): δ=7.42-7.30 (m, 4H), 7.30-7.20 (m, 2H), 7.20-7.07 (m, 2H), 4.10-3.93 (m, 1H), 3.85 (d, J=8.1 Hz, 1H), 3.83-3.74 (m, 1H), 3.71 (d, J=8.4 Hz, OH), 3.64 (p, J=6.9 Hz, 1H), 3.46 (dd, J=8.3, 0.9 Hz, 1H), 3.44-3.37 (m, 1H), 2.99 (p, J=6.8 Hz, OH), 2.75 (dd, J=12.7, 8.7 Hz, OH), 2.34 (dd, J=11.7, 5.4 Hz, 1H), 2.10 (dd, J=11.7, 9.0 Hz, 1H), 1.95 (ddd, J=12.7, 5.0, 0.9 Hz, OH), 1.67 (s, 1H), 1.58 (s, 2H), 1.32-1.27 (m, 4H), 1.27-1.22 (m, 2H), 1.21-1.14 (m, 5H), 1.12 (t, J=6.6 Hz, 3H), 0.97 (d, J=6.0 Hz, 2H).

¹³C{¹H} NMR (100 MHZ, 25° C., CDCl₃): δ=. 2, 151.0, 150.5, 150.2, 150.2, 149.9, 140.6, 139.7, 128.3, 128.2, 126.5, 126.4, 126.0, 125.9, 125.7, 125.7, 124.6, 124.0, 123.7, 123.5, 66.9, 66.8, 58.1, 57.0, 49.1, 46.8, 45.9, 45.3, 31.8, 30.5, 27.9, 27.8, 27.5, 27.4, 25.6, 25.5, 24.8, 24.7, 24.2, 24.2, 23.9, 23.6, 22.3, 20.5.

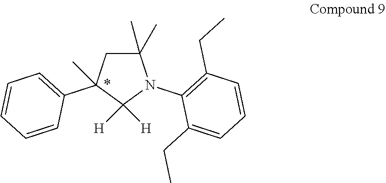 [SMILES: CCc1cccc(CC)c1N1CC(C)(c2ccccc2)CC1(C)C]

¹H NMR (400 MHZ, 25° C., CDCl₃): δ=7.41-7.31 (m, 4H), 7.26-7.21 (m, 1H), 7.17 (d, J=5.3 Hz, 2H), 7.12 (dd, J=5.5, 4.0 Hz, 1H), 3.98 (d, J=8.4 Hz, 1H), 3.49 (d, J=8.3 Hz, 1H), 3.17 (dq, J=15.0, 7.5 Hz, 1H), 2.86-2.65 (m, 2H), 2.60-2.47 (m, 2H), 2.27 (dd, J=12.7, 0.9 Hz, 1H), 1.64 (s, 3H), 1.27-1.20 (m, 6H), 1.19-1.11 (m, 3H), 1.02 (s, 3H).

¹³C{¹H} NMR (100 MHZ, 25° C., CDCl₃): δ=150.7, 147.6, 147.5, 140.3, 128.2, 127.1, 126.6, 126.0, 125.9, 125.6, 65.3, 63.4, 54.5, 45.2, 32.2, 29.9, 29.2, 25.5, 25.4, 16.3, 16.2.

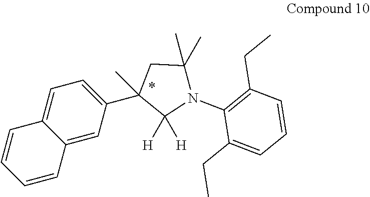 [SMILES: CCc1cccc(CC)c1N1CC(C)(c2ccc3ccccc3c2)CC1(C)C]

¹H NMR (400 MHZ, 25° C., CDCl₃): δ=7.88-7.81 (m, 3H), 7.78 (d, J=2.1 Hz, 1H), 7.54-7.41 (m, 3H), 7.18-7.16 (m, 2H), 7.14-7.10 (m, 1H), 4.09 (d, J=8.6 Hz, 1H), 3.60 (d, J=8.6 Hz, 1H), 3.25-3.12 (m, 1H), 2.89-2.68 (m, 2H), 2.64 (d, J=12.7 Hz, 1H), 2.59-2.45 (m, 1H), 2.36 (d, J=12.7 Hz, 1H), 1.71 (s, 3H), 1.27 (t, J=7.6 Hz, 3H, overlapping) 1.27 (s, 3H, overlapping). 1.14 (t, J=7.5 Hz, 3H), 1.02 (s, 3H).

¹³C{¹H} NMR (100 MHZ, 25° C., CDCl₃): δ=148.0, 147.7, 147.6, 140.5, 133.5, 131.9, 128.0, 128.0, 127.6, 127.2, 126.8, 126.2, 126.1, 125.6, 125.5, 123.8, 65.5, 63.6, 54.8, 45.5, 32.1, 29.9, 29.4, 25.7, 16.5.

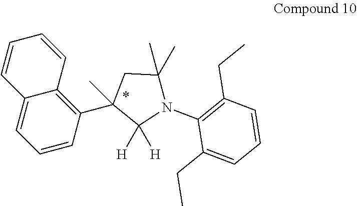 [SMILES: CCc1cccc(CC)c1N1CC(C)(c2cccc3ccccc23)CC1(C)C]

¹H NMR (400 MHZ, 25° C., CDCl₃): δ=8.24-8.18 (m, 1H), 7.92-7.87 (m, 1H), 7.75 (dd, J=6.7, 2.8 Hz, 1H), 7.49-7.42 (m, 4H), 7.21-7.15 (m, 2H), 7.11 (dd, J=6.7, 2.8 Hz, 1H), 4.27 (d, J=8.6 Hz, 1H), 3.85 (d, J=8.6 Hz, 1H), 3.33-3.20 (m, 1H), 2.83-2.70 (m, 2H, overlapping), 2.79 (d, J=12.7 Hz, 1H, overlapping), 2.63 (d, J=12.6 Hz, 1H), 2.53 (dq, J=15.0, 7.6 Hz, 1H), 1.35 (s, 3H), 1.29 (t, J=7.6 Hz, 3H), 1.09 (t, J=7.5 Hz, 3H), 1.04 (s, 3H).

¹³C{¹H} NMR (100 MHZ, 25° C., CDCl₃): δ=147.7, 147.4, 146.9, 140.6, 135.0, 131.6, 129.6, 127.4, 127.3, 126.8, 126.4, 126.2, 125.4, 125.2, 125.0, 123.8, 66.8, 63.0, 56.2, 46.2, 31.3, 30.0, 28.8, 25.7, 25.6, 16.4, 16.3.

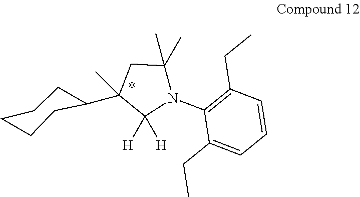 [SMILES: CCc1cccc(CC)c1N1CC(C)(C2CCCCC2)CC1(C)C]

¹H NMR (400 MHZ, 25° C., CDCl₃): 7.16-7.08 (m, 3H), 3.40 (d, J=8.3 Hz, 1H), 3.14-3.05 (m, 1H), 2.98 (d, J=8.3 Hz, 1H), 2.91-2.81 (m, 1H), 2.69-2.58 (m, J=2H), 1.88-1.73 (m, 4H), 1.69 (m, 2H), 1.54-1.46 (m, 1H), 1.30-1.10 (m, 9H, overlapping) 1.21 (s, 3H, overlapping), 1.21 (t, 3H, J=7.6 Hz overlapping), 1.18 (s, 3H, overlapping), 1.17 (t, J=7.5 Hz, 3H, overlapping) 1.04 (s, 3H).

¹³C{¹H} NMR (100 MHZ, 25° C., CDCl₃): δ=147.9, 147.5, 140.9, 127.1, 126.6, 125.9, 66.3, 63.4, 55.3, 50.1, 43.7, 30.3, 29.1, 28.8, 28.6, 27.3, 27.2, 27.0, 25.8, 25.3, 22.3, 16.4, 16.3.

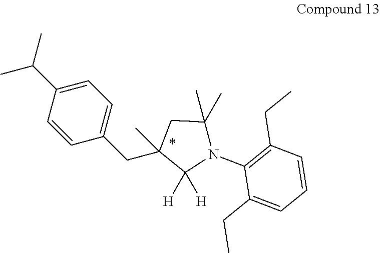 [SMILES: CCc1cccc(CC)c1N1CC(C)(Cc2ccc(C(C)C)cc2)CC1(C)C]

¹H NMR (400 MHZ, 25° C., CDCl₃): δ=7.18-7.10 (m, 7H), 3.51 (d, J=8.5 Hz, 1H), 3.10 (d, J=8.5 Hz, 1H), 3.07-2.95 (m, 2H), 2.96-2.88 (m, 1H), 2.87-2.78 (m, 2H), 2.73-2.56 (m, 2H), 2.08 (d, J=12.8 Hz, 1H), 1.76 (d, J=12.7 Hz, 1H), 1.29-1.23 (m, 12H), 1.17 (t, J=7.6 Hz, 2H, overlapping), 1.16 (s, 3H, overlapping), 1.13 (s, 3H).

¹³C{¹H} NMR (100 MHZ, 25° C., CDCl₃): δ=147.7, 147.4, 146.5, 140.7, 137.2, 130.4, 126.9, 126.8, 126.0, 65.6, 63.6, 54.8, 48.2, 41.7, 33.8, 29.6, 29.3, 27.5, 25.6, 25.5, 24.2, 16.4, 16.4.

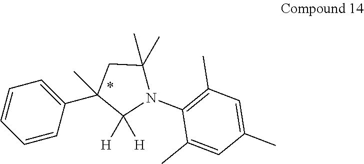 [SMILES: Cc1cc(C)c(N2CC(C)(c3ccccc3)CC2(C)C)c(C)c1]

¹H NMR (400 MHZ, 25° C., CDCl₃): δ=7.42-7.30 (m, 4H), 7.28-7.19 (m, 1H), 6.95 (s, 1H), 6.89 (s, 1H), 4.01 (d, J=8.3 Hz, 1H), 3.41 (d, J=8.3 Hz, 1H), 2.48 (d, 1H, J=11.6 Hz, overlapping), 2.47 (s, 3H), 2.30 (s, 3H), 2.28 (s, 3H), 2.25 (d, J=11.6 Hz, 1H), 1.66 (s, 3H), 1.31 (s, 3H), 1.11 (s, 3H).

¹³C{¹H} NMR (100 MHZ, 25° C., CDCl₃): δ=150.9, 141.3, 140.9, 139.6, 135.0, 130.2, 129.7, 128.3, 126.1, 125.7, 64.1, 63.9, 54.6, 45.0, 32.3, 30.1, 29.5, 21.1, 20.9, 20.9.

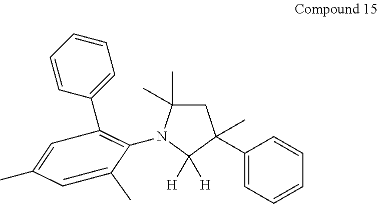 [SMILES: Cc1cc(C)c(N2CC(C)(c3ccccc3)CC2(C)C)c(-c2ccccc2)c1]

Diastereoisomeric ratio of starting iminium salt is 1/1 but product is received in 4/1 mixture.

Analytical data are given for a Major Dia

¹H NMR (400 MHZ, 25° C., CDCl₃): δ=7.48-7.41 (m, 2H), 7.40-7.33 (m, 3H), 7.32-7.26 (m, 2H), 7.26-7.20 (m, 3H), 7.04 (s, 1H), 7.02 (s, 1H), 4.25 (d, J=8.3 Hz, 1H), 3.55 (d, J=8.3 Hz, 1H), 2.37 (s, 3H), 2.35 (s, 3H), 2.30 (d, J=12.6 Hz, 1H), 1.97 (d, J=12.6 Hz, 1H), 1.45 (s, 3H), 1.00 (s, 3H), 0.60 (s, 3H).

¹³C{¹H} NMR (100 MHZ, 25° C., CDCl₃): δ=151.5, 146.2, 144.1, 140.9, 138.5, 135.0, 132.2, 130.4, 130.3, 128.4, 127.5, 126.3, 125.9, 125.7, 65.1, 64.4, 54.1, 45.09, 32.4, 30.5, 28.5, 21.7, 20.9.

### Chiral Resolution: Analytical Chiral HPLC Separation Data (Corresponding to Step b) of the Process According to the Invention for the Preparation of Compounds of Formula (IV) According to the Invention)

### Analytical Chiral HPLC Separation for Compound 1

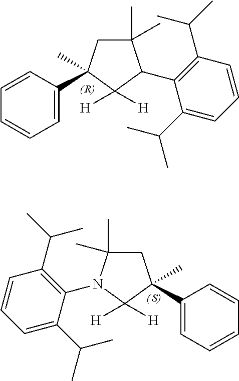

The sample is dissolved in hexane, injected on the chiral column, and detected with an UV detector at 220 nm and a circular dichroism detector at 254 nm. The flow-rate is 1 mL/min.

| | Mobile | | | | | | |
|---|---|---|---|---|---|---|---|
| Column | Phase | t1 | k1 | t2 | k2 | α | Rs |
| Lux-Cellulose-4 | Heptane | 4.57 | 0.55 | 5.54 | 0.88 | 1.60 | 3.15 |
| | | (+) | | (−) | | | |

| RT | | Area | Capacity | | Resolution |
|---|---|---|---|---|---|
| [min] | Area | % | Factor | Enantioselectivity | (USP) |
| 4.57 | 6687 | 49.83 | 0.55 | | |
| 5.54 | 6731 | 50.17 | 0.88 | 1.60 | 3.15 |
| Sum | 13418 | 100.00 | | | |

### Semi-Preparative Separation for Compound 1:

Sample preparation: About 160 mg of compound 1 are dissolved in 1.8 mL of hexane.
Chromatographic conditions: Lux-Cellulose-4 (250×10 mm), hexane as mobile phase, flow-rate=5 mL/min, UV detection at 254 nm.
Injections (stacked): 45 times 40 μL, every 2.8 minutes.
First fraction: 72 mg of the first eluted enantiomer with ee>99.5% (45% yield).

| RT [min] | Area | Area % |
|---|---|---|
| 4.27 | 9480 | 100.00 |
| Sum | 9480 | 100.00 |

Second fraction: 72 mg of the second eluted enantiomer with ee>96% (45% yield).

| RT [min] | Area | Area % |
|---|---|---|
| 4.29 | 368 | 1.97 |
| 4.78 | 18352 | 98.03 |
| Sum | 18720 | 100.00 |

Intermediate: 12 mg

### Optical Rotations

Optical rotations were measured on a Jasco P-2000 polarimeter with a halogen lamp (589, 578 and 546 nm), in a 10 cm cell, thermostated at 25° C. with a Peltier controlled cell holder.

| | (+)-Compound 1 | (−)-Compound 1 |
|---|---|---|
| | First eluted enantiomer | Second eluted enantiomer |
| | on Lux-Cellulose-4 | on Lux-Cellulose-4 |
| λ (nm) | α_{D}²⁵ (CH₂Cl₂, c = 0.62) | α_{D}²⁵ (CH₂Cl₂, c = 0.53) |
| 589 | +61 | −59 |
| 578 | +64 | −62 |
| 546 | +76 | −74 |
| 436 | +152 | −146 |
| 405 | +209 | −201 |
| 365 | +360 | −348 |

### Preparative Separation for Compound 1:

Sample preparation: about 2.54 g of compound 2 are dissolved in 30 ml of hexane.
Chromatographic conditions: Lux-Cellulose-2 (250×10 mm), thermostated at 30° C. in an oven, hexane/2-PrOH (99.9/0.1) as mobile phase, flow-rate=5 mL/min, UV detection at 254 nm
Injections (stacked): 600 times 50 mL, every 1.5 minutes, collection of two fractions.
The first fraction (er 98/2) is dissolved in 16 mL of hexane and was purified again. Injections (stacked): 64 times 250 mL, every 2.5 minutes, to obtain 1.09 g of the first eluted enantiomer ((+)-(R)-Compound 1) with ee>99.5%

| RT [min] | Area | Area % |
|---|---|---|
| 4.67 | 15411 | 100.00 |
| Sum | 15411 | 100.00 |

The second fraction (er 7/93) is dissolved in 14 mL of hexane and was purified again. Injections (stacked): 700 times 20 mL, every 1.5 minutes, to obtain 1.22 g of the second eluted enantiomer ((−)-(S)-Compound 1) with ee>98.5%

| RT [min] | Area | Area % |
|---|---|---|
| 4.78 | 159 | 0.61 |
| 5.58 | 26164 | 99.39 |
| Sum | 26323 | 100.00 |

### Optical Rotations

Optical rotations were measured on a Jasco P-2000 polarimeter with a halogen lamp (589, 578, 546, 436, 405 and 365 nm), in a 10 cm cell, thermostated at 25° C. with a Peltier controlled cell holder.

Structures of (+)-(R)-Compound 1 (from first fraction) and (−)-(S)-Compound 1 (from second fraction) were determined by single crystal X-ray diffraction.

### Analytical Chiral HPLC Separation for Compound 2

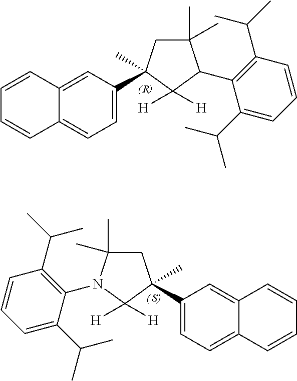

The sample is dissolved in ethanol, injected on the chiral column, and detected with an UV detector at 254 nm. The flow-rate is 0.5 mL/min.

| | Mobile | | | | | | |
|---|---|---|---|---|---|---|---|
| Column | Phase | t1 | k1 | t2 | k2 | α | Rs |
| Lux-Cellulose-3 | Ethanol | 8.14 | 0.3 | 10.95 | 0.75 | 2.49 | 2.87 |

| RT | | Area | Capacity | | Resolution |
|---|---|---|---|---|---|
| [min] | Area | % | Factor | Enantioselectivity | (USP) |
| 8.14 | 4031 | 50.50 | 0.30 | | |
| 10.95 | 3951 | 49.50 | 0.75 | 2.49 | 2.87 |
| Sum | 7982 | 100.00 | | | |

### Preparative Separation for Compound 2

Sample preparation: about 360 mg of compound 2 are dissolved in 150 ml of ethanol.
Chromatographic conditions: Lux-Cellulose-3 (250×10 mm), ethanol as mobile phase, flow-rate=2 mL/min, UV detection at 310 nm.
Injections (stacked): 185 times 800 μL, every 8 minutes.
First fraction: 160 mg of the first eluted enantiomer ((+)—R-compound 2) with ee>99%, 44% yield

| RT [min] | Area | Area % |
|---|---|---|
| 8.20 | 1735 | 100.00 |
| Sum | 1735 | 100.00 |

Second fraction: 160 mg of the second eluted enantiomer ((−)—S-compound 2) with ee>99%, 44% yield

| RT [min] | Area | Area % |
|---|---|---|
| 11.08 | 946 | 100.00 |
| Sum | 946 | 100.00 |

Impurity: 12 mg

### Optical Rotations

Optical rotations were measured on a Jasco P-2000 polarimeter with a halogen lamp (589, 578, 546, 436, 405 and 365 nm), in a 10 cm cell, thermostated at 25° C. with a Peltier controlled cell holder.

| | (+)-Compound 2 | (−)-Compound 2 |
|---|---|---|
| | First eluted enantiomer | Second eluted enantiomer |
| | on Lux-Cellulose-3 | on Lux-Cellulose-3 |
| λ (nm) | α_{D}²⁵ (CH₂Cl₂, c = 1.25) | α_{D}²⁵ (CH₂Cl₂, c = 0.98) |
| 589 | +51 | −51 |
| 578 | +54 | −54 |
| 546 | +64 | −64 |
| 436 | +128 | −128 |
| 405 | +176 | −176 |
| 365 | +300 | −300 |

### Analytical Chiral HPLC Separation for Compound 3

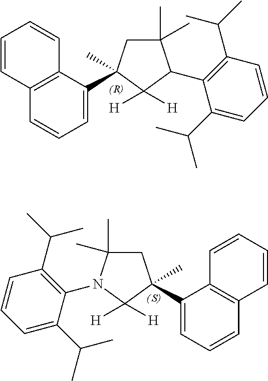

The sample is dissolved in ethanol, injected on the chiral column, and detected with an UV detector at 254 nm. The flow-rate is 0.5 mL/min.

| | Mobile | | | | | | |
|---|---|---|---|---|---|---|---|
| Column | Phase | t1 | k1 | t2 | k2 | α | Rs |
| Lux-Cellulose-3 | Ethanol | 7.76 | 0.24 | 9.62 | 0.54 | 2.23 | 1.41 |

| RT | | Area | Capacity | | Resolution |
|---|---|---|---|---|---|
| [min] | Area | % | Factor | Enantioselectivity | (USP) |
| 7.76 | 5304 | 50.94 | 0.24 | | |
| 9.62 | 5109 | 49.06 | 0.54 | 2.23 | 1.41 |
| Sum | 10412 | 100.00 | | | |

### Preparative Separation for Compound 3

Sample preparation: About 320 mg of compound 3 are dissolved in 15 mL of ethanol.
Chromatographic conditions: Lux-Cellulose-3 (250×10 mm), ethanol as mobile phase, flow-rate=2 mL/min, UV detection at 310 nm.
Injections (stacked): 60 times 250 μL, every 6 minutes.

After collection and evaporation of the first intermediate fraction: 28 times 250 UL, every 5 minutes.

After evaporation of the second intermediate fraction: 20 times 250 μL, every 5 minutes
First fraction: 152 mg of the first eluted enantiomer ((−)—S-compound 3) with ee>99%, 48% yield

| RT [min] | Area | Area % |
|---|---|---|
| 7.81 | 4433 | 100.00 |
| Sum | 4433 | 100.00 |

Second fraction: 152 mg of the second eluted enantiomer ((+)—R-compound 3) with ee>98.5%, 48% yield

| RT [min] | Area | Area % |
|---|---|---|
| 7.77 | 42 | 0.54 |
| 9.71 | 7754 | 99.46 |
| Sum | 7796 | 100.00 |

Impurity: 15 mg

### Optical Rotations

Optical rotations were measured on a Jasco P-2000 polarimeter with a halogen lamp (589, 578, 546, 436, 405 and 365 nm), in a 10 cm cell, thermostated at 25° C. with a Peltier controlled cell holder.

| | (−)-Compound 3 | (+)-Compound 3 |
|---|---|---|
| | First eluted enantiomer | Second eluted enantiomer |
| | on Lux-Cellulose-3 | on Lux-Cellulose-3 |
| λ (nm) | α_{D}²⁵ (CH₂Cl₂, c = 0.77) | α_{D}²⁵ (CH₂Cl₂, c = 0.95) |
| 589 | −44 | 44 |
| 578 | −46 | 46 |
| 546 | −56 | 56 |
| 436 | −118 | 118 |
| 405 | −170 | 170 |
| 365 | −320 | 320 |

### Analytical Chiral HPLC Separation for Compound 4

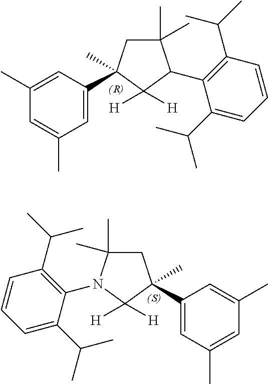

The sample is dissolved in ethanol, injected on the chiral column, and detected with an UV detector at 254 nm and a circular dichroism detector at 254 nm. The flow-rate is 1 mL/min.

| | Mobile | | | | | | |
|---|---|---|---|---|---|---|---|
| Column | Phase | t1 | k1 | t2 | k2 | α | Rs |
| Lux-Cellulose-2 | Heptane/ | 8.75 | 0.40 | 9.70 | 0.55 | 1.38 | 1.72 |
| | 2-PrOH | (+) | | (−) | | | |

| RT | | Area | Capacity | | Resolution |
|---|---|---|---|---|---|
| [min] | Area | % | Factor | Enantioselectivity | (USP) |
| 4.26 | 614 | 53.95 | 0.45 | | |
| 5.02 | 524 | 46.05 | 0.70 | 1.57 | 2.17 |
| Sum | 1137 | 100.00 | | | |

### Semi-Preparative Separation for Compound

Sample preparation: About 234 mg of compound 4 are dissolved in 3.6 mL of hexane.
Chromatographic Lux-Cellulose-2 (250×10 mm), hexane/2-PrOH 99.9/0.1 as mobile phase, flow-rate=5 mL/min, 30° C., UV detection at 290 nm.
Injections (stacked): 90 times 40 μL, every 2.4 minutes.
First fraction: 108 mg of the first eluted enantiomer ((+)-(R)-compound 4) with ee>99.5%

| RT [min] | Area | Area % |
|---|---|---|
| 4.27 | 1645 | 100.00 |
| Sum | 1645 | 100.00 |

Second fraction: (127 mg, er 7/93) is dissolved in 2 mL of hexane and was purified again. Injections (stacked): 50 times 40 μL, every 2.4 minutes, to obtain 106 mg of the second eluted enantiomer ((−)-(S)-compound 4) with ee>98%

| RT [min] | Area | Area % |
|---|---|---|
| 4.31 | 20 | 0.94 |
| 5.01 | 2090 | 99.06 |
| Sum | 2110 | 100.00 |

### Optical Rotations

Optical rotations were measured on a Jasco P-2000 polarimeter with a halogen lamp (589, 578, 546, 436, 405 and 365 nm), in a 10 cm cell, thermostated at 25° C. with a Peltier controlled cell holder.

| | (+)-Compound 4 | (+)-Compound 4 |
|---|---|---|
| | First eluted enantiomer | Second eluted enantiomer |
| | on Lux-Cellulose-2 | on Lux-Cellulose-2 |
| λ (nm) | α_{D}²⁵ (CH₂Cl₂, c = 0.19) | α_{D}²⁵ (CH₂Cl₂, c = 0.18) |
| 589 | +51 | −50 |
| 578 | +54 | −53 |
| 546 | +64 | −63 |
| 436 | +127 | −125 |
| 405 | +177 | −174 |
| 365 | +308 | −302 |
| 325 | +580 | −570 |

### Analytical Chiral HPLC Separation for Compound 6

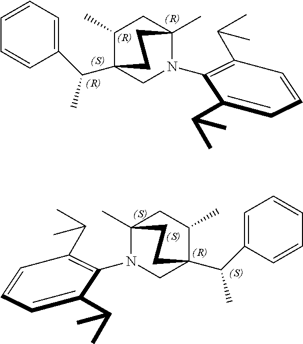

### Analytical Chiral HPLC Separation for Compound 6

The sample is dissolved in heptane/2-PrOH, injected on the chiral column, Lux-Cellulose-2 and detected with an UV detector at 230 nm, a circular dichroism detector at 254 nm. The flow-rate is 1 mL/min.

| RT [min] | Area | Area % | Capacity Factor | |
|---|---|---|---|---|
| 4.44 | 3679 | 39.28 | 0.50 | «Main» 1 |
| 4.81 | 1879 | 20.06 | 0.63 | «Mino» |
| 5.24 | 3807 | 40.65 | 0.78 | «Main 2» |
| Sum | 9364 | 100.00 | | |

### Preparative Separation for Compound 6

Sample preparation: About 100 mg of compound 6 are dissolved in 10 ml of hexane.
Chromatographic conditions: Lux-Cellulose-2 (250×10 mm), hexane/2-PrOH (99.9/0.1) as mobile phase, flow-rate=5 mL/min, UV detection at 290 nm.
Injections (stacked): 200 times 50 μL, every 5 minutes.
First fraction: 25 mg with ee>99.5% (“Main” 1)

| RT [min] | Area | Area % |
|---|---|---|
| 4.45 | 1423 | 100.00 |
| Sum | 1423 | 100.00 |

Second fraction: 14 mg “mino”)

| RT [min] | Area | Area % |
|---|---|---|
| 4.47 | 49 | 8.20 |
| 4.83 | 546 | 91.80 |
| Sum | 595 | 100.00 |

Third fraction: 28 mq with ee>99.5% (“Main” 2)

| RT [min] | Area | Area % |
|---|---|---|
| 5.32 | 486 | 100.00 |
| Sum | 486 | 100.00 |

Intermediate: 17 mg

### Optical Rotations

Optical rotations were measured on a Jasco P-2000 polarimeter with a halogen lamp (589, 578, 546, 436, 405 and 365 nm), in a 10 cm cell, thermostated at 25° C. with a Peltier controlled cell holder.

| | (−)-Compound 6 | (+)-Compound 6 |
|---|---|---|
| | First eluted enantiomer | Second eluted enantiomer |
| | on Lux-Cellulose-2 | on Lux-Cellulose-2 |
| λ (nm) | α_{D}²⁵ (CH₂Cl₂, c = 0.19) | α_{D}²⁵ (CH₂Cl₂, c = 0.18) |
| 589 | −32 | +32 |
| 578 | −33 | +33 |
| 546 | −42 | +42 |
| 436 | −96 | +96 |
| 405 | −145 | +145 |
| 365 | −290 | +290 |

### Analytical Chiral HPLC Separation for Compound 9

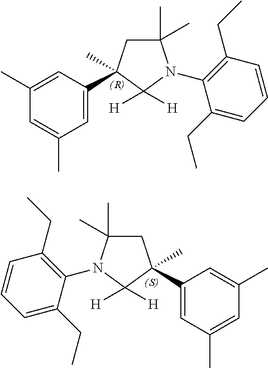

The sample is dissolved in ethanol, injected on the chiral column, and detected with an UV detector at 254 nm and a circular dichroism detector at 254 nm. The flow-rate is 0.5 mL/min.

| | Mobile | | | | | | |
|---|---|---|---|---|---|---|---|
| Column | Phase | t1 | k1 | t2 | k2 | α | Rs |
| Lux-Cellulose-3 | Ethanol | 8.75 | 0.40 | 9.70 | 0.55 | 1.38 | 1.72 |
| | | (+) | | (−) | | | |

| RT | | Area | Capacity | | Resolution |
|---|---|---|---|---|---|
| [min] | Area | % | Factor | Enantioselectivity | (USP) |
| 8.75 | 4714 | 49.91 | 0.40 | | |
| 9.70 | 4731 | 50.09 | 0.55 | 1.38 | 1.72 |
| Sum | 9444 | 100.00 | | | |

### Semi-Preparative Separation for Compound 9:

Sample preparation: About 160 mg of compound 9 are dissolved in 2 mL of ethanol.
Chromatographic conditions: Lux-Cellulose-3 (250×10 mm), ethanol as mobile phase, flow-rate=2 mL/min, UV detection at 254 nm.
Injections (stacked): 25 times 80 μL, every 4 minutes.
First fraction: 69 mg of the first eluted enantiomer ((+)-(R)-compound 9) with ee>99.5% (43% yield)

| RT [min] | Area | Area % |
|---|---|---|
| 8.81 | 18659 | 100.00 |
| Sum | 18659 | 100.00 |

Second fraction: 76 mg of the second eluted enantiomer ((−)-(S)-compound 9) with ee>97% (48% yield)

| RT [min] | Area | Area % |
|---|---|---|
| 8.83 | 299 | 1.46 |
| 9.70 | 20140 | 98.54 |
| Sum | 20439 | 100.00 |

Intermediate: 14 mg

### Optical Rotations

Optical rotations were measured on a Jasco P-2000 polarimeter with a halogen lamp (589, 578, 546, 436, 405 and 365 nm), in a 10 cm cell, thermostated at 25° C. with a Peltier controlled cell holder.

| | (+)-Compound 9 | (−)-Compound 9 |
|---|---|---|
| | First eluted enantiomer | Second eluted enantiomer |
| | on Lux-Cellulose-3 | on Lux-Cellulose-3 |
| λ (nm) | α_{D}²⁵ (CH₂Cl₂, c = 0.19) | α_{D}²⁵ (CH₂Cl₂, c = 0.18) |
| 589 | +64 | −62 |
| 578 | +67 | −65 |
| 546 | +79 | −77 |
| 436 | +157 | −153 |
| 405 | +215 | −209 |
| 365 | +368 | −359 |

### Analytical Chiral HPLC Separation for Compound 13

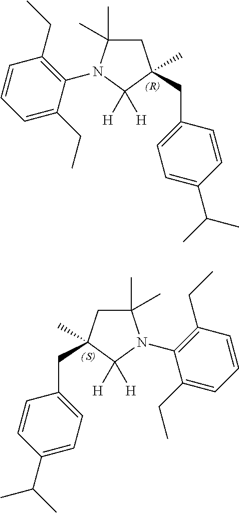 [0254] The sample is dissolved in ethanol, injected on the chiral column Lux-Cellulose-3, and detected with an UV detector at 254 nm. The flow-rate is 0.5 mL/min.

| RT | | Area | Capacity | | Resolution |
|---|---|---|---|---|---|
| [min] | Area | % | Factor | Enantioselectivity | (USP) |
| 8.06 | 3969 | 50.60 | 1.73 | | |
| 8.95 | 3876 | 49.40 | 2.04 | 1.17 | 1.63 |
| Sum | 7845 | 100.00 | | | |

### Preparative Separation for Compound 13:

Sample preparation: About 182 mg of compound 13 are dissolved in 7 mL of ethanol.
Chromatographic conditions: Lux-Cellulose-3 (250×10 mm), methanol as mobile phase, flow-rate=3 mL/min, UV detection at 254 nm.
Injections (stacked): 35 times 200 μL, every 8 minutes.
First fraction: 83 mg of the first eluted enantiomer with ee>99.5%

| RT [min] | Area | Area % |
|---|---|---|
| 8.07 | 2593 | 100.00 |
| Sum | 2593 | 100.00 |

Second fraction: 81 mg of the second eluted enantiomer with ee>99.5%

| RT [min] | Area | Area % |
|---|---|---|
| 7.93 | 6 | 0.25 |
| 8.96 | 2519 | 99.75 |
| Sum | 2526 | 100.00 |

Intermediate: 11 mg

### Optical Rotations

Optical rotations were measured on a Jasco P-2000 polarimeter with a halogen lamp (589, 578, 546, 436, 405 and 365 nm), in a 10 cm cell, thermostated at 25° C. with a Peltier controlled cell holder.

| | (−)-Compound 13 | (+)-Compound 13 |
|---|---|---|
| | First eluted enantiomer | Second eluted enantiomer |
| | on Lux-Cellulose-3 | on Lux-Cellulose-3 |
| λ (nm) | α_{D}²⁵ (CH₂Cl₂, c = 0.19) | α_{D}²⁵ (CH₂Cl₂, c = 0.18) |
| 589 | +4 | −4 |
| 578 | +4 | −4 |
| 546 | +3 | −3 |
| 436 | −3 | +4 |
| 405 | −13 | +13 |
| 365 | −45 | +45 |
| 325 | −135 | +135 |

### Analytical Chiral HPLC Separation for Compound 14

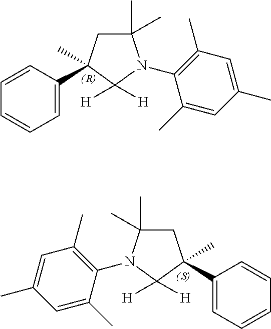 [0263] The sample is dissolved in heptane/2-PrOH, injected on the chiral column Lux-Cellulose-2, and detected with an UV detector at 230 nm and a polarimetric detector. The flow-rate is 1 mL/min.

| RT | | Area | Capacity | | Resolution |
|---|---|---|---|---|---|
| [min] | Area | % | Factor | Enantioselectivity | (USP) |
| 5.43 | 9568 | 49.06 | 0.84 | | |
| 7.20 | 9936 | 50.94 | 1.44 | 1.72 | 4.10 |
| Sum | 19504 | 100.00 | | | |

### Preparative Separation for Compound 14:

Sample preparation: About 127 mg of compound 14 are dissolved in 8 mL hexane
Chromatographic conditions: Lux-Cellulose-2 (250×10 mm), hexane/2-PrOH (99.9/0.1) as mobile phase, flow-rate=5 mL/min, UV detection at 290 nm.
Injections (stacked): 45 times 180 μL, every 5.25 minutes.
First fraction: 54 mg with ee>99.5%

| RT [min] | Area | Area % |
|---|---|---|
| 5.44 | 6293 | 100.00 |
| Sum | 6293 | 100.00 |

Second fraction: 53 mg with ee>99.5%

| RT [min] | Area | Area % |
|---|---|---|
| 5.56 | 13 | 0.14 |
| 7.20 | 9150 | 99.86 |
| Sum | 9163 | 100.00 |

### Optical Rotations

Optical rotations were measured on a Jasco P-2000 polarimeter with a halogen lamp (589, 578, 546, 436, 405 and 365 nm), in a 10 cm cell, thermostated at 25° C. with a Peltier controlled cell holder.

| | (−)-Compound 14 | (+)-Compound 14 |
|---|---|---|
| | First eluted enantiomer | Second eluted enantiomer |
| | on Lux-Cellulose-2 | on Lux-Cellulose-2 |
| λ (nm) | α_{D}²⁵ (CH₂Cl₂, c = 0.15) | α_{D}²⁵ (CH₂Cl₂, c = 0.16) |
| 578 | −84 | +84 |
| 546 | −99 | +99 |
| 436 | −192 | +192 |
| 405 | −260 | +260 |
| 365 | −430 | +430 |
| 325 | −135 | +135 |

### Analytical Chiral HPLC Separation for Compound 15

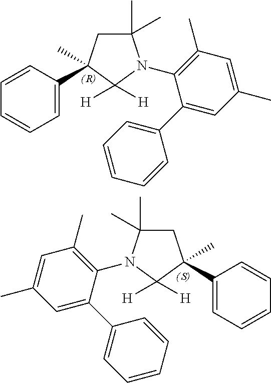 [0271] The sample is dissolved in heptane/2-PrOH, injected on the chiral column, Lux-Cellulose-2 and detected with an UV detector at 230 nm and a polarimetric detector. The flow-rate is 1 mL/min.

| RT | | Area | Capacity | | Resolution |
|---|---|---|---|---|---|
| [min] | Area | % | Factor | Enantioselectivity | (USP) |
| 8.78 | 26945 | 50.19 | 1.98 | | |
| 11.78 | 26742 | 49.81 | 2.99 | 1.51 | 3.21 |
| Sum | 53687 | 100.00 | | | |

### Preparative Separation for Compound 15:

Sample preparation: About 80 mg of compound 15 are dissolved in 10 mL of hexane.
Chromatographic conditions: Lux-Cellulose-2 (250×10 mm), hexane/2-PrOH (99.9/0.1) as mobile phase, flow-rate=5 mL/min, UV detection at 290 nm.
Injections (stacked): 67 times 150 μL, every 9 minutes.
First fraction: 38 mg with ee>99.5%

| RT [min] | Area | Area % |
|---|---|---|
| 8.73 | 27988 | 100.00 |
| Sum | 27988 | 100.00 |

Second fraction: 41 mg with ee>99.5%

| RT [min] | Area | Area % |
|---|---|---|
| 9.09 | 18 | 0.14 |
| 11.93 | 13268 | 99.86 |
| Sum | 13286 | 100.00 |

### Optical Rotations

Optical rotations were measured on a Jasco P-2000 polarimeter with a halogen lamp (589, 578, 546, 436, 405 and 365 nm), in a 10 cm cell, thermostated at 25° C. with a Peltier controlled cell holder.

| | (−)-Compound 15 | (+)-Compound 15 |
|---|---|---|
| | First eluted enantiomer | Second eluted enantiomer |
| | on Lux-Cellulose-2 | on Lux-Cellulose-2 |
| λ (nm) | α_{D}²⁵ (CH₂Cl₂, c = 0.16) | α_{D}²⁵ (CH₂Cl₂, c = 0.21) |
| 589 | −76 | +76 |
| 578 | −80 | +80 |
| 546 | −95 | +95 |
| 436 | −190 | +190 |
| 405 | −262 | +262 |
| 365 | −410 | +410 |

CAAC-H₂ Adduct Oxidation to Obtain the CAAC·BF₄ Iminium Salt (Corresponding to Step c) of the Process According to the Invention for the Preparation of Compounds of Formula (I) According to the Invention)

General procedure: In a Schlenk tube under argon enantiopure CAAC-H₂ adducts were dissolved in dry DCM. Received solution was then cooled down in an ice bath to 0° C. and bromine (3 equiv) was added dropwise. Reaction mixture was then brought to RT and stirred overnight. Then water solution of KBF₄ (6 equiv) and Na₂S₂O₃ (3 equiv) was then added and resulting biphasic mixture was stirred for an hour. Phases were then separated and water phase was additionally washed with extra DCM. Combined organic phases were dried over anhydrous MgSO₄ and filtered. Remaining solution was then reduced to ca. 5 ml and an excess of Et₂O was added causing precipitation of white solid. Filtration and copious washing of the precipitate with Et₂O and pentane afforded cyclic iminium salt BF 4 in typical yield of 85% as white solids.

Structures of Compounds Isolated and Analysed by NMR for which Study of Single Crystals by Means of X-Ray Diffractometry Allowed for Determination of Absolute Configuration.

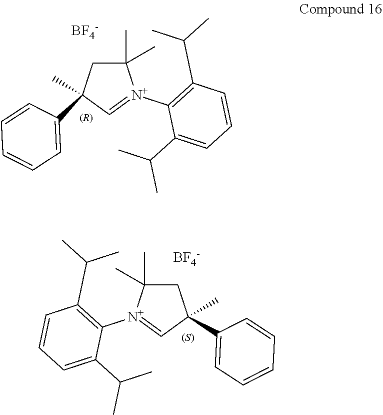

(−)-(R)-Compound-16 (received from (+)-(R)-Compound 1) and its enantiomer (+)-(S)-Compound-16 (received from (−)-(S)-Compound 1) have identical spectra

¹H NMR (500 MHZ, 25° C., CD₃CN): δ: 9.26 (s, 1H), 7.64 (t, J=7.5 Hz 1H), 7.55 (t, J=7.5 Hz, 2H), 7.52 (d, J=7.5 Hz, 1H), 7.48 (d, J=7.5 Hz, 2H), 7.45 (d, J=7.5 Hz, 2H), 3.10 (d, J=14.0 Hz, 1H), 2.82 (d, J=14.0 Hz, 1H), 2.79 (sept, J=7.0 Hz, 1H), 2.55 (sept, J=7.0 Hz, 1H), 1.93 (s, 3H), 1.58 (s, 3H), 1.40 (s, 3H), 1.39 (d, J=7.0 Hz, 3H), 1.25 (d, J=7.0 Hz, 3H), 1.15 (d, J=7.0 Hz, 3H), 1.08 (d, J=7.0 Hz, 3H).

¹³C NMR (125 MHZ, CD₃CN): δ: 189.8, 145.7, 145.4, 142.0, 133.2, 130.8, 130.0, 129.6, 126.7, 126.6, 126.6, 85.3, 55.7, 48.6, 29.9, 29.7, 27.2, 26.8, 26.8, 25.6, 25.5, 21.5, 21.4.

¹¹B NMR (128 MHZ, CDCl₃): δ: −0.98.

¹⁹F NMR (376 MHZ, CDCl₃): δ: −151.0 (small), 151.1

Compounds have opposite [α]_{D}=
(−)-(R)-Compound-16 (T=25° C., c=0.110 g/mL, L=10 cm, CHCl₃)=−22.0
(+)-(S)-Compound-16 (T=25° C., c=0.110 g/mL, L=10 cm, CHCl₃)=+22.1

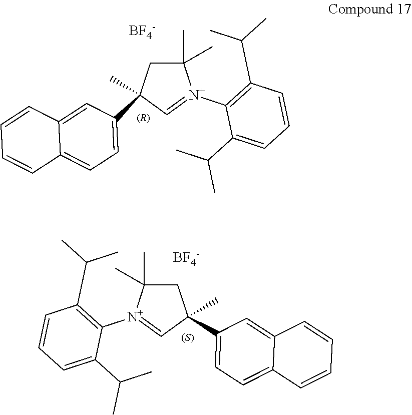

(−)-(R)-Compound-17 (received from (+)-(R)-Compound 2) and its enantiomer (+)-(S)-Compound-17 (received from (−)-(S)-Compound 2) have identical spectra.

¹H NMR (300 MHZ, CD₃CN) δ: ppm) 9.72 (s, 1H), 8.06 (d, J=8.1 Hz, 1H), 7.89-7.97 (m, 3H), 7.58-7.66 (m, 4H), 7.51 (d, J=8.1 Hz, 1HHz), 7.47 (d, J=8.1 Hz, 1H), 3.22 (d, J=14.1 Hz, 1H), 2.88 (d, J=14.1 Hz, 1H), 2.83 (sept, J=6.6 Hz, 1H), 2.60 (sept, J=6.6 Hz, 1H), 2.01 (s, 3H), 1.60 (s, 3H), 1.41 (d, J=6.6 Hz, 3H), 1.40 (s, 3H), 1.21 (d, J=6.6 Hz, 3H), 1.19 (d, J=6.6 Hz, 3H), 1.15 (d, J=6.6 Hz, 3H)

¹³C NMR (125 MHz, CD₃CN) δ: 190.4, 145.7, 145.4, 139.7, 134.2, 133.7, 133.1, 130.8, 130.1, 128.9, 128.7, 128.2, 128.1, 126.7, 126.5, 125.6, 124.4, 85.3, 55.9, 48.5, 29.9, 29.7, 27.4, 26.9, 26.9, 25.7, 25.6, 21.6, 21.5.

¹¹B NMR (128 MHZ, CDCl₃) δ: −0.91.

¹⁹F NMR (376 MHZ, CDCl₃): δ: −150.9 (small), −151.0.

Compounds have opposite [α]_{D}=
(−)-(S)-Compound-16 (T=25° C., c=0.110 g/mL, L=10 cm, CHCl₃)=−239.2
(+)-(R)-Compound-16 (T=25° C., c=0.110 g/mL, L=10 cm, CHCl₃)=+241.8

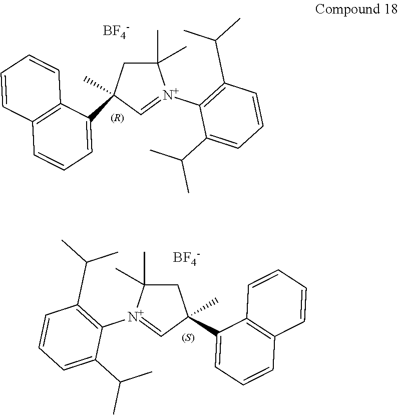

(+)-(R)-Compound-18 (received from (+)-(R)-Compound 3) and its enantiomer

(−)-(S)-Compound-18 (received from (−)-(S)-Compound 3) have identical spectra:

¹H NMR (400 MHZ, CDCl₃) δ: 9.89 (s, 1H), 8.03 (d, J=8.0 Hz, 1H), 7.91 (d, J=8.0 Hz, 1H), 7.80 (d, J=8.0 Hz, 1H), 7.61 (t, J=7.5 Hz, 1H), 7.52-7.55 (m, 2H), 7.33-7.38 (m, 4H), 3.30 (d, J=14.0 Hz, 1H), 3.19 (d, J=14.0 Hz, 1H), 2.70 (sept, J=6.5 Hz, 1H), 2.69 (sept, J=6.5 Hz, 1H), 2.15 (s, 3H), 1.54 (s, 3H), 1.37 (d, J=6.5 Hz, 3H), 1.31 (d, J=6.5 Hz, 3H), 1.25 (s, 3H), 1.23 (d, J=6.5 Hz, 3H), 1.20 (d, J=6.5 Hz, 3H).

¹³C NMR (125 MHZ, CDCl₃) δ: 191.6, 145.3, 144.3, 138.3, 135.8, 132.6, 130.5, 130.1, 129.3, 129.3, 127.2, 126.6, 126.0, 126.0, 125.6, 124.8, 123.5, 84.0, 55.8, 50.0, 30.0, 29.4, 28.1, 26.9, 26.9, 25.9, 25.6, 22.3, 22.0.

¹¹B NMR (128 MHZ, CDCl₃) δ: −0.91.

¹⁹F NMR (376 MHZ, CDCl₃) δ: ppm) −150.9 (small), −151.0.

Compounds have opposite [α]_{D}=
(−)-(S)-Compound-18 (T=25° C., c=0.110 g/mL, L=10 cm, CHCl₃)=−21.3
(+)-(R)-Compound-18 (T=25° C., c=0.110 g/mL, L=10 cm, CHCl₃)=+22.2

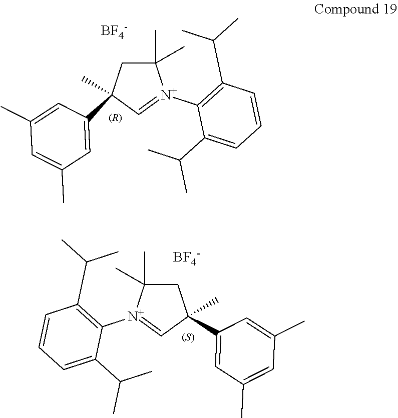

(−)-(R)-Compound-19 (received from (+)-(R)-Compound 4) and its enantiomer (+)-(S)-Compound-19 (received from (−)-(S)-Compound 4) have identical spectra:

¹H NMR (500 MHZ, CDCl₃) δ: 9.59 (s, 1H), 7.51 (t, J=7.5 Hz, 1H), 7.34 (d, J=7.5 Hz, 1H), 7.29 (d, J=7.5 Hz, 1H), 7.06 (s, 2H), 6.97 (s, 1H), 3.16 (d, J=14.0 Hz, 1H), 2.67 (sept, J=6.5 Hz, 1H), 2.66 (d, J=14.0 Hz, 1H), 2.39 (sept, J=6.5 Hz, 1H), 2.31 (s, 6H), 1.87 (s, 3H), 1.52 (s, 3H), 1.35 (d, J=6.5 Hz, 3H), 1.31 (s, 3H), 1.18 (d, J=6.5 Hz, 3H), 1.16 (d, J=6.5 Hz, 3H), 1.12 (d, J=6.5 Hz, 3H).

¹³C NMR (125 MHZ, CDCl₃) δ: 191.0, 145.2, 144.6, 141.2, 140.1, 132.4, 130.5, 129.3, 125.8, 123.6, 83.6, 55.3, 48.5, 30.0, 29.1, 28.7, 27.0, 26.4, 25.9, 25.7, 22.2, 21.9, 21.2.

¹¹B NMR (128 MHz, CDCl₃) δ: −0.99.

¹⁹F NMR (376 MHZ, CDCl₃): δ: (ppm) −151.2 (small), −151.3.

Compounds have opposite [α]_{D}=
(−)-(S)-Compound-19 (T=25° C., c=0.110 g/mL, L=10 cm, CHCl₃)=−71.4
(+)-(R)-Compound-19 (T=25° C., c=0.110 g/mL, L=10 cm, CHCl₃)=+71.5

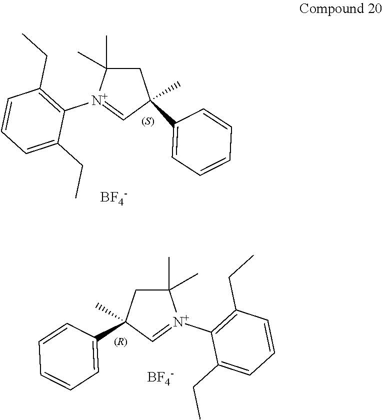

(−)-(R)-Compound-20 (received from (+)-(R)-Compound 9) and its enantiomer (+)-(S)-Compound-20 (received from (−)-(S)-Compound 9) have identical spectra:

¹H NMR (500 MHZ, CDCl₃) δ: 9.55 (s, 1H), 7.47 (t, J=7.5 Hz, 2H), 7.44 (d, J=7.5 Hz, 2H), 7.42 (d, J=7.5 Hz, 1H), 7.33 (t, J=7.5 Hz, 1H), 7.31 (d, J=7.5 Hz, 1H), 7.24 (d, J=7.5 Hz, 1H), 3.16 (d, J=14.0 Hz, 1H), 2.67 (d, J=14.0 Hz, 1H), 2.55 (q, J=7.5 Hz, 2H), 2.33 (dt, J=7.5 Hz, 1H), 2.16 (dt, J=7.5 Hz, 1H), 1.91 (s, 3H), 1.52 (s, 3H), 1.31 (s, 3H), 1.26 (t, J=7.5 Hz, 3H), 1.09 (t, J=7.5 Hz, 3H).

¹³C NMR (125 MHZ, CDCl₃) δ: 190.5, 141.0, 140.2, 139.7, 131.8, 131.0, 130.3, 128.9, 128.3, 128.1, 126.0, 83.8, 55.5, 48.3, 28.9, 26.9, 26.6, 24.8, 24.6, 15.3, 14.5.

¹¹B NMR (128 MHZ, CDCl₃) δ: −0.98.

¹⁹F NMR (376 MHZ, CDCl₃): δ: −151.0 (small), −151.1

Compounds have opposite [α]_{D}=
(−)-(S)-Compound-20 (T=25° C., c=0.110 g/mL, L=10 cm, CHCl₃)=−59.7
(+)-(R)-Compound-20 (T=25° C., c=0.110 g/mL, L=10 cm, CHCl₃)=+60.3

Structures of (+)-(R)-Compound 16, (+)—S-Compund-16, (−)-(R)-Compound 17, (+)—S-Compound 18, (−)—R-Compound 18, (−)-(R)-Compound 19 and (−)-(R)-Compound 20 were determined by single crystal X-ray diffraction.

Structures of compounds isolated and analysed by NMR for which enantiomers were assigned as (+) or (−) based on sign of their optical rotation.

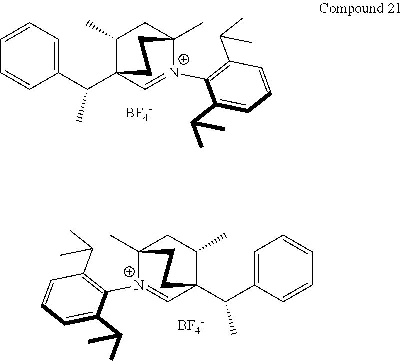

(+)-Compound 21 and (−)-Compound 21 were received from (+) or (−)-Compound 6

¹H NMR (400 MHZ, 25° C., acetone-d₆): δ=8.82 (s, 1H), 7.48 (t, J=7.8 Hz, 1H), 7.42-7.29 (m, 5H), 7.27-7.22 (m, 1H), 7.18 (dd, J=7.8, 1.5 Hz, 1H), 3.77 (q, J=7.2 Hz, 1H), 3.47 (q, J=7.0 Hz, 1H), 2.93-2.79 (m, 2H), 2.72 (m, 1H), 2.67-2.60 (m, 1H), 2.51-2.40 (m, 1H), 1.76-1.58 (m, 5H), 1.47 (d, J=7.2 Hz, 3H), 1.38 (d, J=6.8 Hz, 3H), 1.24 (d, J=6.7 Hz, 3H), 1.22-1.18 (m, 7H), 0.34 (d, J=6.8 Hz, 3H).

¹³C{¹H} NMR (100 MHZ, 25° C., acetone-d₆): δ=190.6, 143.7, 143.5, 141.0, 135.7, 132.1, 129.7, 129.3, 128.0, 125.7, 125.7, 70.5, 66.0, 51.9, 44.0, 39.0, 35.9, 33.0, 30.2, 28.9, 25.6, 25.4, 24.1, 23.4, 22.6, 21.7, 20.5, 15.4, 13.5.

¹¹B NMR (128 MHZ, CDCl₃) δ: −0.90.

¹⁹F NMR (376 MHZ, CDCl₃): δ: −152.7 (small), −152.8

(−)-Compound-21 (T=25° C., c=0.101 g/mL, L=10 cm, acetonitrile)=−87.4

(+)-Compound-21 (T=25° C., c=0.103 g/mL, L=10 cm, acetonitrile)=+87.1

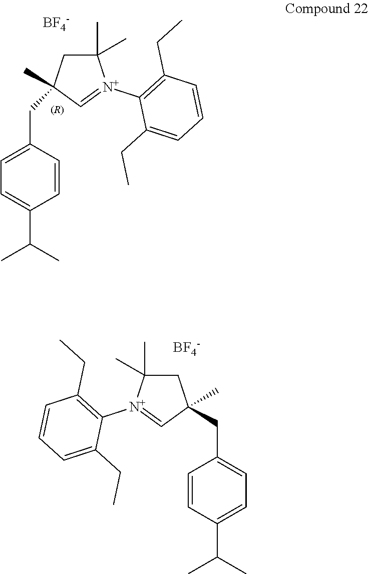

(+)-Compound 22 and (−)-Compound 22 were received from (+) or (−)-Compound 13.

¹H NMR (400 MHZ, 25° C., CDCl₃): δ=9.38 (s, 1H), 7.45 (t, J=7.7 Hz, 1H), 7.35-7.29 (m, 3H), 7.27-7.21 (m, 3H), 3.75 (d, J=14.0 Hz, 1H), 2.93 (sept, J=6.9 Hz, 1H), 2.86 (d, J=14.0 Hz, 1H), 2.72 (d, J=13.7 Hz, 1H), 2.51 (q, J=7.5 Hz, 2H), 2.27 (d, J=13.7 Hz, 1H), 1.89-1.78 (m, 5H), 1.45 (s, 3H), 1.32 (d, J=7.4 Hz, 3H), 1.26 (dd, J=6.9, 1.8 Hz, 6H), 1.11 (t, J=7.5 Hz, 3H), 0.98 (s, 3H).

¹³C{¹H} NMR (100 MHZ, 25° C., CDCl₃): δ=192.5, 148.4, 139.8, 139.4, 133.4, 131.2, 130.5, 130.4 (2C), 127.6 (2C), 127.2 (2C), 83.0, 54.5, 44.4, 43.6, 33.8, 28.1, 27.9, 27.4, 24.7, 24.4, 24.0, 24.0, 15.3, 14.9.

¹¹B NMR (128 MHZ, CDCl₃) δ: −0.89.

¹⁹F NMR (376 MHZ, CDCl₃): δ: −151.1 (small), 151.2.

(−)-Compound-22 (T=25° C., c=0.120 g/mL, L=10 cm, CHCl₃)=−65.7

(+)-Compound-22 (T=25° C., c=0.110 g/mL, L=10 cm, CHCl₃)=+64.8

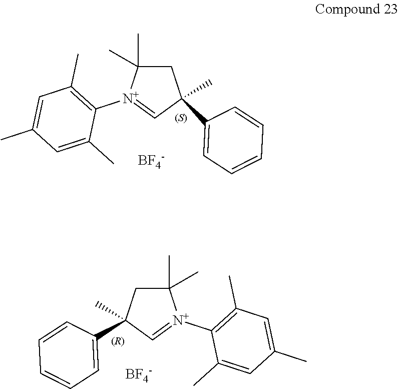

(+)-Compound 23 and (−)-Compound 23 were received from (+) or (−)-Compound 14.

¹H NMR (400 MHZ, 25° C., acetone-d₆): δ=9.71 (s, 1H), 7.65 (m, 2H), 7.50 (m, 2H), 7.44-7.35 (m, 1H), 7.18 (s, 1H), 7.14 (s, 1H), 3.23 (d, J=13.9 Hz, 1H), 2.94 (d, J=14.1 Hz, 1H), 2.37 (s, 3H), 2.31 (s, 3H), 2.17 (s, 3H), 2.01 (s, 3H, overlapping with acetone), 1.70 (s, 3H), 1.52 (s, 3H).

¹³C{¹H} NMR (100 MHZ, 25° C., acetone-d₆): δ=190.8, 142.4, 134.8, 134.4, 131.2, 131.1, 130.5, 129.1, 126.7, 85.6, 56.1, 49.1, 28.6, 27.7, 27.4, 20.7, 19.4.

¹¹B NMR (128 MHz, acetone-d₆) δ: −0.93.

¹⁹F NMR (376 MHZ, acetone-d₆ δ: −151.2 (small), −151.3

(−)-Compound-23 (T=25° C., c=0.120 g/mL, L=10 cm, acetonitrile)=−67.5

(+)-Compound-23 (T=25° C., c=0.122 g/mL, L=10 cm, acetonitrile)=+68.3

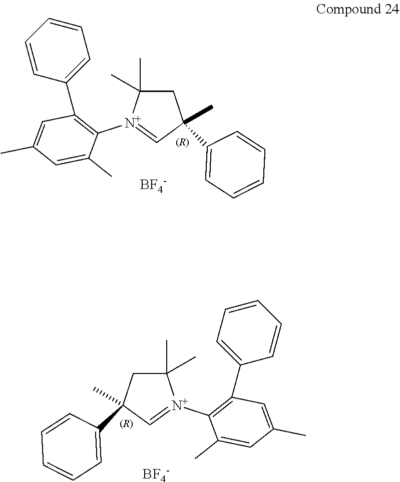

(+)-Compound 24 and (−)-Compound 24 were received from (+) or (−)-Compound 15.

Diastereoisomeric ratio of starting amine is 4/1 and iminium salt is received in same ratio

¹H NMR (400 MHZ, 25° C., CDCl₃): δ=9.85 (s, 1H), 7.53 (m, 2H), 7.47-7.28 (m, 7H), 7.20-6.95 (m, 3H), 2.96 (d, J=13.9 Hz, 1H), 2.36 (s, 3H), 2.09 (s, 3H), 2.06 (d, J=13.9 Hz, 1H), 1.76 (s, 3H), 1.23 (s, 3H), 0.81 (s, 3H). (analytical data given for major isomer, aromatic region is hard to define as signals for two diastereomers overlap).

¹³C{¹H} NMR (100 MHz, 25° C., acetone-d₆): δ=191.5, 191.4, 142, 7, 142.6, 141.9, 141.2, 139.4, 139.3, 139.1, 138.7, 135.7, 135.5, 132.9, 132.9, 131.9, 131.8, 131.1, 130.7, 130.7, 130.6, 130.1, 129.7, 129.4, 129.4, 127.3, 126.9, 85.9, 85.7, 56.3, 56.1, 49.3, 48.8, 29.0, 28.7, 27.9, 27.6, 27.3, 27.1, 21.0, 19.5, 19.5. (analytical data given for mixture of diasteroisomers)

¹¹B NMR (128 MHZ, acetone-d₆) δ: −0.89

¹⁹F NMR (376 MHZ, acetone-d₆): δ: −151.2 (small), −151.3

(−)-Compound-24 (T=25° C., c=0.141 g/mL, L=10 cm, acetonitrile)=−40.1

(+)-Compound-24 (T=25° C., c=0.136 g/mL, L=10 cm, acetonitrile)=+39.3

### Complexation of CAAC Iminium Salt to Prove the Ee Conservation

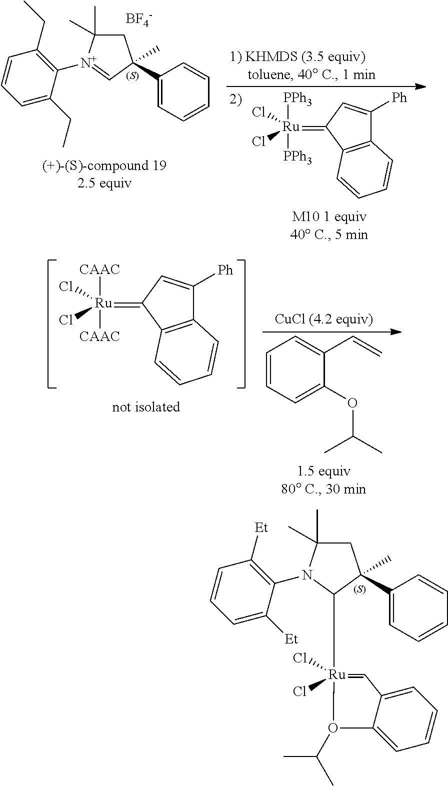

Procedure for the (−)-(S)—Ru complex: In a glove box, (+)-(S)-compound 19 (2.5 equiv) was dissolved in dry and degassed Toluene (0.5 mL). KHMDS (0.5 M in Toluene, 2.5 equiv) was added. The mixture was allowed to stirred 1 min at 40° C. Then, M10 catalyst (1 equiv) and toluene (0.5 mL) were then added. The mixture was stirred 5 min at 40° C. CuCl (4.5 equiv), Styrenyl ether (1.6 equiv) and Toluene (0.5 mL) were added. The mixture was stirred at 80° C. for 30 min out of the box. Volatiles were removed under vacuum and the product was purified by column chromatography (eluent: toluene). Green fraction was washed with pentane.

The desired complex is obtained as a green solid (61% yield) as a mixture of rotamers (ratio determined by ¹H NMR in CDCl₃: 76:24).

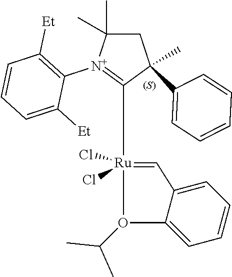

¹H NMR (400 MHz, CDCl₃): δ 17.78 (s, 0.23H), 16.45 (s, 0.77H), 8.45 (d, J=9.1 Hz, 1H), 8.23 (s, 1H), 7.75-7.38 (m, 8H), 6.98 (d, J=9.0 Hz, 1H), 5.15-4.97 (m, 1H), 3.30-3.07 (m, 1H), 2.86-2.64 (m, 2H), 2.63-2.48 (m, 2H), 2.48-2.24 (m, 4H), 1.69-1.50 (m, 6H), 1.50-1.28 (m, 8H), 1.19-1.01 (m, 3H), 0.98-0.74 (m, 3H).

¹³C NMR (101 MHZ, CDCl₃): δ: 295.1, 260.6, 156.5, 143.5, 143.2, 142.6, 138.2, 132.1, 129.5, 129.4, 128.7, 128.6, 127.6, 127.4, 127.1, 125.4, 118.2, 113.2, 78.4, 63.2, 48.4, 31.1, 29.7, 27.6, 25.6, 24.2, 22.2, 14.8, 14.3.

[α]_{D}=(−)-(S)-ruthenium complex (T=25° C., c=0.110 g/mL, L=10 cm, CH₂Cl₂)=−565.

Analytical data for this compound were consistent with the previously reported data.

### Analytical Chiral HPLC for (−)-(S)—Ru Complex

The sample is dissolved in dichloromethane, injected on the chiral column Chiralpak IE, and detected with an UV detector at 254 nm and a circular dichroism detector at 254 nm. The flow-rate is 1 mL/min, Heptane/Ethanol/dichloromethane (60/20/20)

ee determination: 98%

| RT [min] | Area | Area % |
|---|---|---|
| 4.98 | 13 | 1.02 |
| 5.87 | 1294 | 98.98 |
| Sum | 1308 | 100.00 |

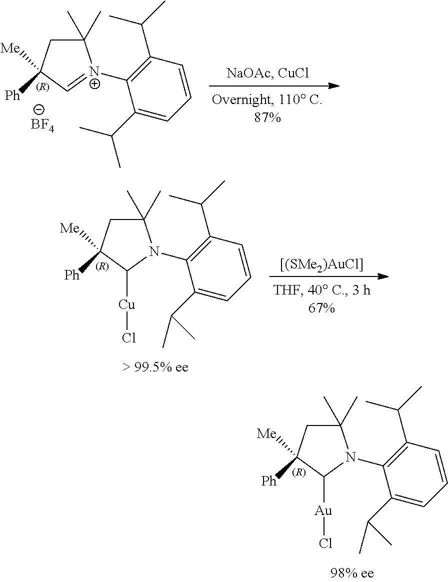

### Procedure for the Au and Cu Complexes Preparation:

In a glovebox, a 100 mL Schlenk flask equipped with a magnetic stirring bar and a septum was charged with (+)-(R)-Compound-14 (100.0 mg, 0.23 mmol, 1.0 eq), copper (I) chloride (25.0 mg, 0.25 mmol, 1.1 eq) and sodium acetate (56.5 mg, 0.69 mmol, 3.0 eq). Toluene (11 mL) was added, and the reaction vessel was brought outside of a glovebox. Septum was then change for a glass stopcock with a metal clipper and the reaction mixture was stirred overnight at 110° C. in a close system. After cooling down to RT, the suspension was opened to air, filtered through a silica gel column, and washed with dichloromethane. The pure (+)-(R)-copper complex (86.7 mg, 0.2 mmol) was obtained as a white powder (Isolated mass=86.7 mg, Yield=87%)

¹H NMR (400 MHZ, CDCl₃) δ: 7.57-7.46 (m, 2H), 7.46-7.31 (m, 3H), 7.31-7.19 (m, 3H), 2.86 (m 2H), 2.58 (d, J=13.4 Hz, 1H), 2.32 (d, J=13.4 Hz, 1H), 1.82 (s, 3H), 1.38-1.30 (m, 12H), 1.26 (d, J=6.7 Hz, 3H), 1.21 (s, 3H).

¹³C NMR (101 MHZ, CDCl₃): δ: ¹³C NMR (101 MHZ, CDCl₃) δ 246.8, 145.9, 145.1, 144.8, 134.5, 129.9, 129.0, 127.2, 126.3, 124.9, 124.9, 81.2, 60.9, 51.4, 29.2, 29.2, 28.2, 28.1, 27.2, 27.2, 22.5, 22.4.

[α]_{D}=(+)-(R)-copper complex (T=25° C., c=0.110 g/mL, L=10 cm, CH₂Cl₂)=+20

### Analytical Chiral HPLC for Cu Complex

ee determination: The sample is dissolved in dichloromethane, injected on the chiral column Chiralpak IG, and detected with an UV detector at 254 nm and a circular dichroism detector mL/min at 254 nm. The flow-rate is 1 mL/min Heptane/Isopropanol/dichloromethane (80/10/10), 1 mL/min. ee>99.5%

| RT [min] | Area | Area % |
|---|---|---|
| 10.88 | 7752 | 100.00 |
| Sum | 7752 | 100.00 |

In a glovebox, a Schenck was charged with the (R)-copper complex (23.6 mg, 0.055 mmol, 1.0 eq), [(SMe2)AuCl] (24.2 mg, 0.082 mmol, 1.5 eq), and THF (0.5 mL). The mixture was then heated at 40 degrees for 4 hours. Solvent was removed under reduced pressure and the crude was purified by column chromatography (dichloromethane). The received solid was filtered over packed celite to remove any nanoparticles. (R)-gold complex (19.9 mg, 0.035 mmol) was obtained as a white solid (19.9 mg, 64% yield).

¹H NMR (400 MHZ, CD₂Cl₂): δ: 7.59-7.53 (m, 2H), 7.51 (d, J=7.7 Hz, 1H), 7.48-7.39 (m, 2H), 7.39-7.29 (m, 3H), 2.96 (hept, J=6.8 Hz, 1H), 2.84 (hept, J=6.7 Hz, 1H), 2.65 (d, J=13.4 Hz, 1H), 2.45 (d, J=13.4 Hz, 1H), 1.92 (s, 3H), 1.46 (d, J=2.4 Hz, 3H), 1.45 (d, J=2.4 Hz, 3H), 1.42 (s, 3H), 1.36 (d, J=6.7 Hz, 3H), 1.33 (d, J=6.8 Hz, 3H), 1.29 (s, 3H).

¹³C NMR (101 MHZ, CD₂Cl₂) δ: 234.6, 145.8, 145.6, 145.3, 134.7, 130.5, 129.3, 127.7, 126.9, 125.6, 125.4, 81.1, 61.4, 52.4, 29.8, 29.6, 28.9, 28.6, 28.5, 27.1, 26.9, 23.1, 22.8.

[α]_{D}=(+)-(R)-gold complex (T=25° C., c=0.153 g/mL, L=10 cm, acetonitrile)=+24

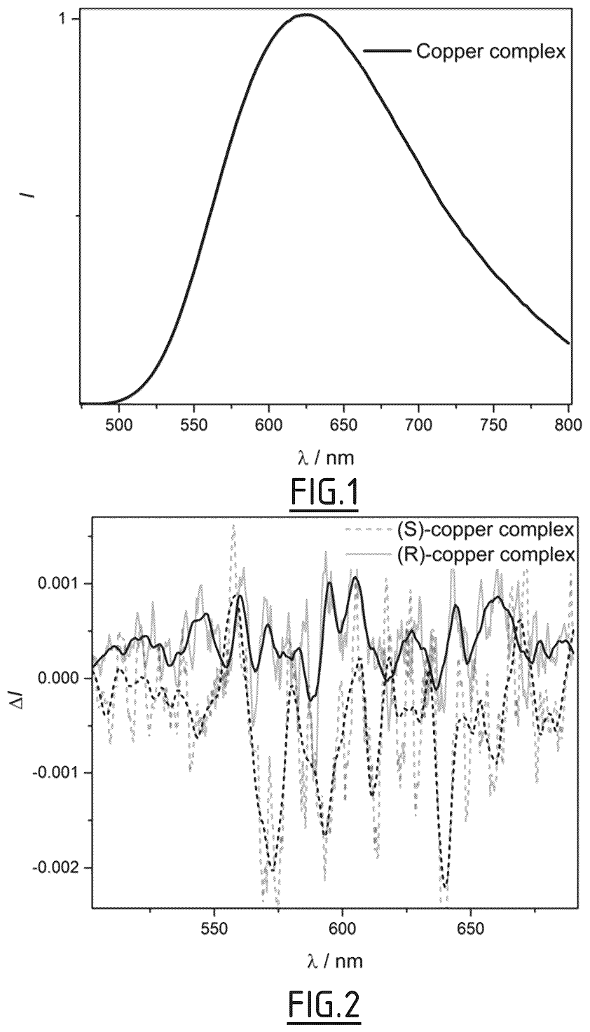

As preliminary photophysical and chiroptical characterizations, the unpolarized (black solid line) and circularly polarized luminescence (CPL) of the enantiopure copper complexes ((R) and(S), blue and red solid lines, respectively, with an average gium value of 10⁻³) were measured using a CPL spectrofluoropolarimeter. The samples were excited using a 90° geometry with a Xenon ozone-free lamp 150 W LS. The following parameters were used: emission slit width≈2 mm, integration time=4 sec, scan speed=50 nm/min, accumulations=5. The concentration of all the samples was ˜10⁻⁵ M. Excitation of the samples were performed at 320 nm. The corresponding results are shown in FIGS. 1 and 2.

### Analytical Chiral HPLC for Au Complex

ee determination: The sample is dissolved in dichloromethane, injected on the chiral column Chiralpak IG, and detected with an UV detector at 254 nm and a circular dichroism detector at 254 nm. The flow-rate is 1 mL/min Heptane/Isopropanol/dichloromethane (80/10/10), 1 mL/min. ee>98%

| RT [min] | Area | Area % |
|---|---|---|
| 10.33 | 7583 | 99.21 |
| 19.46 | 60 | 0.79 |
| Sum | 7643 | 100.00 |

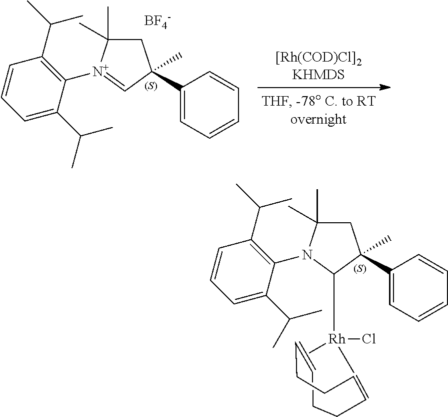

### Procedure for the (+)-(S)—Rh Complex:

[Rh(COD)Cl]₂ (36.3 mg, 0.074 mmol, 0.5 equiv.), (−)-(S)-Compound-14 (75 mg, 0.172 mmol, 1.2 equiv.) and KHMDS (39.2 mg, 0.197 mmol, 1.3 equiv.) were added to a Schlenk tube in the glovebox. Out of the box under Ar atmosphere, dry and degassed THF (3 mL) was added dropwise over 10 min to the solids at −78° C. The suspension was stirred for 10 min at −78° C., after which the cooling bath was removed, and the reaction mixture was allowed to warm up to rt. After stirring for 16 h at room temperature, volatiles were removed under vacuum. The product was purified by column chromatography (pentane/diethyl ether=9:1) to received yellow-orange solid of (+)-(S)—Rh complex (44.9 mg 51% yield)

¹H NMR (400 MHZ, CDCl₃): 7.99 (d, J=7.3 Hz, 2H), 7.41 (dt, J=17.2, 7.6 Hz, 4H), 7.29 (d, J=7.2 Hz, 1H), 7.11 (dd, J=7.5, 1.8 Hz, 1H), 5.32 (m, 1H), 4.52 (m, 1H), 3.87 (m, 1H), 2.86 (d, J=13.3 Hz, 1H), 2.65 (m, 2H), 2.51-2.40 (m, 1H), 2.38 (s, 3H), 2.07 (d, J=13.2 Hz, 1H), 1.91 (m, 1H), 1.75 (d, J=6.4 Hz, 3H), 1.66 (s, 3H), 1.66-1.49 (m, 3H), 1.48-1.38 (m, 2H), 1.35 (s, 3H), 1.29 (two pairs of d overlapping, 6H), 1.24-1.07 (m, 2H), 0.72 (d, J=6.7 Hz, 3H).

¹³C NMR (101 MHZ, CDCl₃): 269.23 (d, J=46.7 Hz), 147.98, 146.11, 145.89, 136.57, 129.00, 128.50, 128.11, 126.59, 126.53, 124.15, 101.81 (d, J=5.8 Hz), 98.00 (d, J=6.4 Hz), 78.67, 78.65, 72.11 (d, J=14.6 Hz), 66.37 (d, J=14.0 Hz), 49.03, 35.16, 33.29, 31.28, 30.16, 28.84, 28.58, 28.56, 26.27, 25.52, 25.28, 24.58.

[α]_{D}=(+)-(S)-Rhodium complex (T=25° C., c=0.110 g/mL, L=10 cm, CH₂Cl₂)=+5

### Analytical Chiral HPLC for Rh Complex

ee determination: The sample is dissolved in dichloromethane, injected on the chiral column Chiralpak IB N-5, and detected with an UV detector at 254 nm and a circular dichroism detector at 254 nm. The flow-rate is 1 mL/min. ee>99%

| RT [min] | Area | Area % |
|---|---|---|
| 6.80 | 1386 | 100.00 |
| Sum | 1386 | 100.00 |

1. A process for the preparation of an optically pure (+) or (−) enantiomer of an iminium salt having the following formula (I): 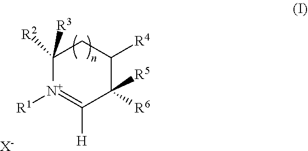
wherein:
R¹ is a (C₆-C₁₄)aryl group, a (C₁-C₆)alkyl group or a (C₈-C₂₀)cycloalkyl group,
said aryl group being optionally substituted with at least one substituent chosen selected from the group consisting of: halogen, (C₆-C₁₀)aryl group, and (C₁-C₆)alkyl group, said alkyl group being optionally substituted with one or several phenyl group(s); or
R¹ is a —NR′ₐR′_{b} group, R′ₐ and R′_{b} being independently from each other selected from the group consisting of: H, (C₁-C₆)alkyl, and (C₆-C₁₀)aryl, or R′ₐ and R′_{b} form together with the nitrogen atom carrying them a N(CH₂)₂₊ₘ heterocyclyl ring, m being 0 or an integer comprised from 1 to 6;
R² is H, a (C₆-C₁₀)aryl group or a (C₁-C₆)alkyl group;
R³ is a (C₁-C₆)alkyl group; or R² and R³ may together form, with the carbon atom carrying them, a (C₃-C₆)cycloalkyl;
R⁵ is selected from the following groups: (C₆-C₂₀)aryl, (C₁-C₁₀)alkyl, and (C₃-C₁₂)cycloalkyl group,
said alkyl group being optionally substituted with at least one substituent selected from the (C₆-C₁₀)aryl groups, and
said aryl group being optionally substituted with at least one substituent selected from the group consisting of: (C₁-C₆)alkyl, optionally substituted with one or several phenyl group(s), (C₆-C₁₀)aryl(C₁-C₆)alkyl, and (C₆-C₁₀)aryl, optionally substituted with one or several substituents selected from the group consisting of: (C₁-C₆)alkylamino, di(C₁-C₆)alkylamino, (C₁-C₆) alkoxy, and (C₁-C₆)alkyl;
R⁶ is selected from the following groups: (C₆-C₂₀)aryl, (C₁-C₁₀)alkyl, (C₃-C₁₂)cycloalkyl, heteroaryl, (C₆-C₁₀)aryl(C₁-C₆)alkyl, and heteroaryl(C₁-C₆)alkyl,
said aryl group being optionally substituted with at least one substituent selected from the group consisting of: (C₁-C₆)alkyl, optionally substituted with one or several phenyl group(s), (C₆-C₁₀)aryl(C₁-C₆)alkyl, and (C₆-C₁₀)aryl, optionally substituted with one or several substituents selected from the group consisting of: (C₁-C₆)alkylamino, di(C₁-C₆)alkylamino, (C₁-C₆) alkoxy, and (C₁-C₆)alkyl;
with the proviso that R⁶ is different from R⁵; or
R⁵ and R⁶, taken together with the carbon atom to which they are attached, form a five-, six-, or ten-membered cycloalkyl or heterocyclyl ring;
R⁴ is H or a (C₁-C₆)alkyl group;
n is 0 or an integer comprised between 1 and 3; or R³ and R⁵, taken together with the carbon atom to which they are attached, form a six-, seven- or eight-membered cycloalkyl ring;
X⁻ is a counteranion,
said salt being in the form of an optically pure (+) or (−) enantiomer,
said process comprising the following steps of:
a) reducing an iminium salt having the following formula (II),
said salt being in the form of a racemic mixture: 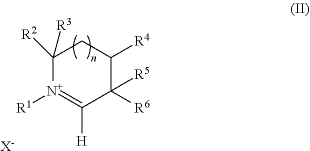
R¹, R², R³, R⁴, R⁵, R⁶, n, and X⁻ are as defined above in formula (I),
in order to obtain a compound having the formula (III): 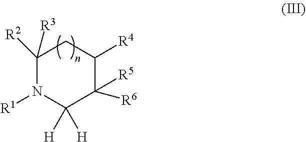
R¹, R², R³, R⁴, R⁵, R⁶, and n are as defined above in formula (I),
said compound of formula (III) being in the form of a racemic mixture,
b) separating, by chiral HPLC, the compound of formula (III) in the form of a racemic mixture, for obtaining an optically pure (+) or (−) enantiomer compound having the formula (IV): 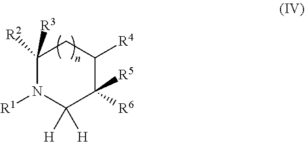
R¹, R², R³, R⁴, R⁵, R⁶, and n are as defined above in formula (I),
said compound of formula (IV) being in the form of an optically pure (+) or (−) enantiomer,
c) oxidizing the compound of formula (IV) for obtaining the compound of formula (I), and
d) optionally a counteranion exchange step.

2. The process of claim 1, wherein the reducing step is carried out with a reduction agent selected from the group consisting of: LiAlH₄, NaBH₄, diisobutylaluminium hydride, lithium triethylborohydride, sodium bis(2-methoxyethoxy)aluminium hydride, and cyanoborohydrides.

3. The process of claim 1, wherein the oxidizing step is carried out with a oxidation agent selected from the group consisting of: Br₂, N-bromosuccinimide, I₂, N-iodosuccinimide, Cl₂, a copper (II) compound, and a hypervalent iodine compound.

4. The process of claim 1, wherein R¹ is a (C₆-C₁₀)aryl group substituted with at least one substituent selected from (C₁-C₆)alkyl groups, and/or wherein R² is a (C₁-C₆)alkyl group.

5. The process of claim 1, wherein R² and R³ are identical.

6. The process of claim 1, wherein R² and R³ are different.

7. The process of claim 1, wherein R⁴ is H.

8. The process of claim 1, wherein R⁵ and R⁶ are different and selected from the following groups: a (C₆-C₁₀)aryl group, a (C₁-C₆)alkyl group, such as methyl, and a (C₃-C₆)cycloalkyl group, such as cyclohexyl, said aryl group being optionally substituted with two substituents selected from the (C₁-C₆)alkyl groups.

9. An optically pure (+) or (−) enantiomer of an iminium salt having the following formula (I): 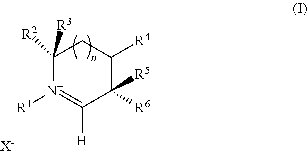
wherein:
R¹ is a (C₆-C₁₄)aryl group, a (C₁-C₆)alkyl group or a (C₈-C₂₀)cycloalkyl group, said aryl group being optionally substituted with at least one substituent selected from the group consisting of: halogen, a (C₆-C₁₀)aryl group, and a (C₁-C₆)alkyl group, said alkyl group being optionally substituted with one or several phenyl group(s); or
R¹ is a —NR′ₐR′_{b} group, R′ₐ and R′_{b} being independently from each other selected from the group consisting of: H, (C₁-C₆)alkyl, and (C₆-C₁₀)aryl, or Ra and Rb form together with the nitrogen atom carrying them a N(CH₂)₂₊ₘ heterocyclyl ring, m being 0 or an integer comprised from 1 to 6;
R² is H, a (C₆-C₁₀)aryl group or a (C₁-C₆)alkyl group;
R³ is a (C₁-C₆)alkyl group; or R² and R³ may together form, with the carbon atom carrying them, a (C₃-C₆)cycloalkyl;
R⁵ is selected from the following groups: (C₆-C₂₀)aryl, (C₁-C₁₀)alkyl, and (C₃-C₁₂)cycloalkyl group,
said alkyl group being optionally substituted with at least one substituent selected from the (C₆-C₁₀)aryl groups, and said aryl group being optionally substituted with at least one substituent selected from the group consisting of: (C₁-C₆)alkyl, optionally substituted with one or several phenyl group(s), (C₆-C₁₀)aryl(C₁-C₆)alkyl, and (C₆-C₁₀)aryl, optionally substituted with one or several substituents selected from the group consisting of: (C₁-C₆)alkylamino, di(C₁-C₆)alkylamino, (C₁-C₆) alkoxy, and (C₁-C₆)alkyl;
R⁶ is selected from the following groups: (C₆-C₂₀)aryl, (C₁-C₁₀)alkyl, (C₃-C₁₂)cycloalkyl, heteroaryl, (C₆-C₁₀)aryl(C₁-C₆)alkyl, and heteroaryl(C₁-C₆)alkyl,
said aryl group being optionally substituted with at least one substituent selected from the group consisting of: (C₁-C₆)alkyl, optionally substituted with one or several phenyl group(s), (C₆-C₁₀)aryl(C₁-C₆)alkyl, and (C₆-C₁₀)aryl, optionally substituted with one or several substituents selected from the group consisting of: (C₁-C₆)alkylamino, di(C₁-C₆)alkylamino, (C₁-C₆) alkoxy, and (C₁-C₆)alkyl;
with the proviso that R⁶ is different from R⁵; or
R⁵ and R⁶, taken together with the carbon atom to which they are attached, form a five-, six-, or ten-membered cycloalkyl or heterocyclyl ring;
R⁴ is H or a (C₁-C₆)alkyl group;
n is 0 or an integer comprised between 1 and 3; or
R³ and R⁵, taken together with the carbon atom to which they are attached, form a six-, seven- or eight-membered cycloalkyl ring; and
X⁻ is a counteranion,
said salt being in the form of an optically pure (+) or (−) enantiomer.

10. (canceled)

11. A combination comprising the compound of formula (I) as defined in claim 1, and a transition metal other than ruthenium.

12. A combination comprising the compound of formula (I) as defined in claim 1, and a transition metal selected from the group consisting of gold, copper, and rhodium.

13. A combination comprising the compound of formula (I) as defined in claim 1, a transition metal, and an organic light-emitting diode.

14. The combination of claim 13, wherein the transition metal is selected from the group consisting of gold, copper, and rhodium.

15. An organic light emitting device (OLED) comprising: an anode; a cathode; and an organic layer disposed between the anode and the cathode,
wherein the organic layer comprises a compound of formula (I) according to claim 1, in combination with a transition metal selected from the group consisting of Ru, Os, Ir, Pd, Pt, Cu, Ag, and Au, and can be bonded to other ligands.

16. The process of claim 3, wherein the oxidation agent is selected from the group consisting of 2-iodoxybenzoic acid, 2,3-dichloro-5,6-dicyano-1,4-benzoquinone, tetra-N-butylammonium iodide, and tert-butyl hydroperoxide.

17. The process of claim 1, wherein R¹ is a (C₆-C₁₀)aryl group substituted with at least one substituent selected from a phenyl group substituted with two alkyl groups, optionally wherein the alkyl groups are methyl, isopropyl or ethyl groups, and R¹ is a methyl group.

18. The process of claim 5, wherein R² and R³ are a methyl group.

19. The process of claim 6, wherein R² is a (C₁-C₆)alkyl group or a methyl group, and R³ is H, a (C₆-C₁₀)aryl group, or a phenyl group.

20. The process of claim 1, wherein R⁵ and R⁶ are different and selected from the following groups: a phenyl or naphthyl, a methyl, and a cyclohexyl.

21. The optically pure (+) or (−) enantiomer of an iminium salt according to claim 9, wherein n is 1.
